SEQUENCE LISTING (24 sequences)

SEQ ID NO: 1 - DNA, 2573 bp, Homo sapiens
gggtggcgccgggagtgggaacgccacacagtgccaaatccccggctccagctcccgactcccggctcccggctcccggctcccggtgcccaatcccgggccgcagccatgaacggcgaggagcagtactacgcggccacgcagctttacaaggacccatgcgcgttccagcgaggcccggcgccggagttcagcgccagcccccctgcgtgcctgtacatgggccgccagcccccgccgccgccgccgcacccgttccctggcgccctgggcgcgctggagcagggcagccccccggacatctccccgtacgaggtgccccccctcgccgacgaccccgcggtggcgcaccttcaccaccacctcccggctcagctcgcgctcccccacccgcccgccgggcccttcccggagggagccgagccgggcgtcctggaggagcccaaccgcgtccagctgcctttcccatggatgaagtctaccaaagctcacgcgtggaaaggccagtgggcaggcggcgcctacgctgcggagccggaggagaacaagcggacgcgcacggcctacacgcgcgcacagctgctagagctggagaaggagttcctattcaacaagtacatctcacggccgcgccgggtggagctggctgtcatgttgaacttgaccgagagacacatcaagatctggttccaaaaccgccgcatgaagtggaaaaaggaggaggacaagaagcgcggcggcgggacagctgtcgggggtggcggggtcgcggagcctgagcaggactgcgccgtgacctccggcgaggagcttctggcgctgccgccgccgccgccccccggaggtgctgtgccgcccgctgcccccgttgccgcccgagagggccgcctgccgcctggccttagcgcgtcgccacagccctccagcgtcgcgcctcggcggccgcaggaaccacgatgagaggcaggagctgctcctggctgaggggcttcaaccactcgccgaggaggagcagagggcctaggaggaccccgggcgtggaccacccgccctggcagttgaatggggcggcaattgcggggcccaccttagaccgaaggggaaaacccgctctctcaggcgcatgtgccagttggggccccgcgggtagatgccggcaggccttccggaagaaaaagagccattggtttttgtagtattggggccctcttttagtgatactggattggcgttgtttgtggctgttgcgcacatccctgccctcctacagcactccaccttgggacctgtttagagaagccggctcttcaaagacaatggaaactgtaccatacacattggaaggctccctaacacacacagcggggaagctgggccgagtaccttaatctgccataaagccattcttactcgggcgacccctttaagtttagaaataattgaaaggaaatgtttgagttttcaaagatcccgtgaaattgatgccagtggaatacagtgagtcctcctcttcctcctcctcctcttccccctccccttcctcctcctcctcttcttttccctcctcttcctcttcctcctgctctcctttcctccccctcctcttttccctcctcttcctcttcctcctgctctcctttcctccccctcctctttctcctcctcctcctcttcttccccctcctctccctcctcctcttcttccccctcctctccctcctcctcttcttctccctcctcttcctcttcctcctcttccacgtgctctcctttcctccccctcctcttgctccccttcttccccgtcctcttcctcctcctcctcttcttctccctcctcttcctcctcctctttcttcctgacctctttctttctcctcctcctccttctacctccccttctcatccctcctcttcctcttctctagctgcacacttcactactgcacatcttataacttgcacccctttcttctgaggaagagaacatcttgcaaggcagggcgagcagcggcagggctggcttaggagcagtgcaagagtccctgtgctccagttccacactgctggcagggaaggcaaggggggacgggcctggatctgggggtgagggagaaagatggacccctgggtgaccactaaaccaaagatattcggaactttctatttaggatgtggacgtaattcctgttccgaggtagaggctgtgctgaagacaagcacagtggcctggtgcgccttggaaaccaacaactattcacgagccagtatgaccttcacatctttagaaattatgaaaacgtatgtgattggagggtttggaaaaccagttatcttatttaacattttaaaaattacctaacagttatttacaaacaggtctgtgcatcccaggtctgtcttcttttcaaggtctgggccttgtgctcgggttatgtttgtgggaaatgcttaataaatactgataatatgggaagagatgaaaactgattctcctcactttgtttcaaacctttctggcagtgggatgattcgaattcacttttaaaattaaattagcgtgttttgttttg

SEQ ID NO: 2 - protein, 283 aa, Homo sapiens
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 3 - DNA, 19 bp, Artificial Sequence
  Description of Artificial Sequence Synthetic primer
cgcgcttcttgtcctcctc

SEQ ID NO: 4 - DNA, 15 bp, Artificial Sequence
  Description of Artificial Sequence Synthetic primer
gtaggcgccgcctgc

SEQ ID NO: 5 - DNA, 20 bp, Artificial Sequence
  Description of Artificial Sequence Synthetic primer
aggttttcgggcaccactta

SEQ ID NO: 6 - DNA, 20 bp, Artificial Sequence
  Description of Artificial Sequence Synthetic primer
tgatgttgctgtccccaagt

SEQ ID NO: 7 - DNA, 21 bp, Artificial Sequence
  Description of Artificial Sequence Synthetic primer
tcccagctcagcatgaaagct

SEQ ID NO: 8 - DNA, 21 bp, Artificial Sequence
  Description of Artificial Sequence Synthetic primer
aaaaccgctgatcacgctctg

SEQ ID NO: 9 - DNA, 20 bp, Artificial Sequence
  Description of Artificial Sequence Synthetic primer
ccactgtggaggaccagaat

SEQ ID NO: 10 - DNA, 20 bp, Artificial Sequence
  Description of Artificial Sequence Synthetic primer
ctcgtgaggcctcttacctg

SEQ ID NO: 11 - DNA, 18 bp, Artificial Sequence
  Description of Artificial Sequence Synthetic primer
ttgtgtccaccttccaca

SEQ ID NO: 12 - DNA, 18 bp, Artificial Sequence
  Description of Artificial Sequence Synthetic primer
gctgtccaagttgctcat

SEQ ID NO: 13 - DNA, 20 bp, Artificial Sequence
  Description of Artificial Sequence Synthetic primer
catcaaccggcttaatggtg

SEQ ID NO: 14 - DNA, 20 bp, Artificial Sequence
  Description of Artificial Sequence Synthetic primer
actttcacacgcaggatgga

SEQ ID NO: 15 - DNA, 25 bp, Artificial Sequence
  Description of Artificial Sequence Synthetic primer
gaagttcatctgcaccaccggcaag

SEQ ID NO: 16 - DNA, 23 bp, Artificial Sequence
  Description of Artificial Sequence Synthetic primer
ggtagctcaggtagtggttgtcg

SEQ ID NO: 17 - DNA, 20 bp, Artificial Sequence
  Description of Artificial Sequence Synthetic primer
cgcgccatttcggacccgga

SEQ ID NO: 18 - DNA, 22 bp, Artificial Sequence
  Description of Artificial Sequence Synthetic primer
cgtcaaggcgctcttgcctacg

SEQ ID NO: 19 - DNA, 25 bp, Artificial Sequence
  Description of Artificial Sequence Synthetic primer
atgttccagtatgactccactcacg

SEQ ID NO: 20 - DNA, 25 bp, Artificial Sequence
  Description of Artificial Sequence Synthetic primer
gaagacaccagtagactccacgaca

SEQ ID NO: 21 - DNA, 20 bp, Artificial Sequence
  Description of Artificial Sequence Synthetic primer
cgcgccatttcggacccgga

SEQ ID NO: 22 - DNA, 22 bp, Artificial Sequence
  Description of Artificial Sequence Synthetic primer
cgtcaaggcgctcttgcctacg

SEQ ID NO: 23 - DNA, 20 bp, Artificial Sequence
  Description of Artificial Sequence Synthetic primer
ggtggagctggctgtcatgt

SEQ ID NO: 24 - DNA, 21 bp, Artificial Sequence
  Description of Artificial Sequence Synthetic primer
aagtctaacaaagctcacgcg

(19) United States
(12) Patent Application Publication
(10) Pub. No.: US 2015/0017638 A1
(43) Pub. Date: Jan. 15, 2015

(54) METHODS FOR ASSESSING RISK FOR CANCER USING BIOMARKERS

(71) Applicant: THE TRUSTEES OF THE UNIVERSITY OF PENNSYLVANIA, Philadelphia, PA (US)

(72) Inventors: ANDREW D. RHIM, Philadelphia, PA (US); Ben Z. Stanger, Merion Station, PA (US)

(73) Assignee: THE TRUSTEES OF THE UNIVERSITY OF PENNSYLVANIA, Philadelphia, PA (US)

(21) Appl. No.: 14/335,820

(22) Filed: Jul. 18, 2014

Related U.S. Application Data
(63) Continuation of application No. PCT/US2013/022227, filed on Jan. 18, 2013.
(60) Provisional application No. 61/587,969, filed on Jan. 18, 2012.
(60) Provisional application No. 61/660,297, filed on Jun. 15, 2012.

(51) Int. Cl. G01N 33/574; G01N 33/68; C12Q 1/68; G01N 33/569; G01N 33/50; C07K 16/18

(52) U.S. Cl. CPC G01N 33/57438; G01N 33/57496; G01N 33/5011; C07K 16/18; C12Q 1/6886; C12Q 1/6883; G01N 33/56966; G01N 33/5044; G01N 33/6872; G01N 2800/50; G01N 2800/56; G01N 2800/7095
USPC 435/6.11; 435/34; 435/7.1; 530/387.9; 435/7.92; 435/7.94; 435/6.12

(57) ABSTRACT

The present disclosure is directed to methods and kits for detecting, characterizing, preventing, and treating cancer, e.g., pancreatic cancer, or inflammation, by determining the presence of circulating epithelial in a biological sample and further identifying the tissue of origin using a tissue-specific biomarker, e.g., Pdx-1.

### CROSS-REFERENCE TO RELATED APPLICATIONS

This application is a continuation of International Application No. PCT/US13/022,227, filed Jan. 18, 2013, which claims priority to U.S. Provisional Patent Application Ser. No. 61/587,969, filed on Jan. 18, 2012, and U.S. Provisional Patent Application Ser. No. 61/660,297, filed on Jun. 15, 2012, each of which is incorporated by reference herein in its entirety, and priority to each of which is claimed.

### GRANT INFORMATION

This invention was made with government support under NIH K08-DK088945 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Jul. 18, 2014, is named 08140601.txt and is 11,213 bytes in size.

### BACKGROUND

Cancer is one of the leading causes of death in the developed world, resulting in over 500,000 deaths per year in the United States alone. Over one million people are diagnosed with cancer in the U.S. each year, and overall it is estimated that more than one in three people will develop some form of cancer during their lifetime. Pancreatic ductal adenocarcinoma has a median survival of 6 months and a five year survival of less than 5%, making it one of the most lethal human cancers (Warshaw, A. L. and C. Fernandez-del Castillo (1992) N Engl J Med 326: 455-65).

Most cancer patients are not killed by their primary tumor. Instead, cancer patients succumb to metastases: the spread of malignant cells from one part of the body to another. If a primary tumor is detected early enough, it can often be eliminated by surgery, radiation, chemotherapy or some combination of these treatments. In contrast, metastatic tumors are difficult to detect and treatment becomes more challenging as metastases progresses. As such, there is a need to develop methods for detecting early-stage cancer metastasis and for understanding the process of metastasis.

Each step in the metastatic cascade is highly inefficient. Only a small fraction of cells from a primary tumor enter the circulation as circulating tumor cells (CTCs), and less than 0.01% of these develop into metastases (Gupta et al., 2005). It is thought that tumor cells pass through several stages during which they sequentially acquire the ability to invade through basement membrane(s), enter and exit the bloodstream, and survive and grow in distant organs. Because each of these events is rare, studies of the metastatic process have relied heavily upon cells that have been cultured and manipulated in vitro and reintroduced into recipient animals. As a result, there remains considerable uncertainty regarding the factors that influence each stage in vivo as well as the timing of dissemination itself.

Clinical observations, mainly in the field of breast cancer, have given rise to two major metastasis paradigms. The classical model treats metastasis as the final step in a progressive “Darwinian” sequence, in which tumors acquire mutations that promote invasive behavior and dissemination late in tumor evolution (Cairns, 1975). This model has several conceptual problems (Gupta et al., 2005; Klein, 2009) and fails to account for two clinical observations: the appearance of metastatic lesions years after resection of small tumors with no clinically evident metastases at diagnosis (Pantel et al., 2008) and metastases of unknown primary tumors, which account for as many as 4%-5% of all clinical metastases (Greco and Hainsworth, 2009).

An alternative model has been proposed that envisions metastasis as an inherent feature of a tumor very early in its natural history (Hellman, 1994; Klein, 2009). Although direct evidence for this model is limited, recent studies of breast cancer are consistent with the notion that metastatic seeding may be mediated by cells that would not meet a standard definition of cancer (Husemann et al., 2008; Podsypanina et al., 2008). Furthermore, several small studies concluded that the presence of putative disseminated tumor cells in the bone marrow of patients with low-grade mammary tumors or carcinoma in situ correlates with a worse outcome (Ignatiadis et al., 2011; Stanger et al., 2011). The possibility that cellular dissemination leading to metastasis occurs prior to the formation of an identifiable primary tumor has significant clinical and biological implications.

One of the challenges in studying circulating tumor cell (CTC) dissemination has been the identification of markers that distinguish cancer cells from cells that normally reside in the bloodstream or at sites of seeding. During malignant progression, it has been proposed that carcinoma cells undergo an epithelial-to-mesenchymal transition (EMT), in which they lose epithelial characteristics and acquire invasive properties and stem cell-like features (Polyak and Weinberg, 2009). Although several studies support a physiologic role during tumor progression (Moody et al., 2005; Trimboli et al., 2008), most studies of EMT in the context of cancer biology have been conducted in vitro, and thus the relevance of EMT to carcinogenesis continues to be debated (Ledford, 2011).

Moreover, standard techniques for isolating CTCs rely heavily upon the use of epithelial markers, particularly EpCAM, for detection (Pantel et al., 2008). However, detection methods that rely on cellular expression of epithelial markers alone, including EpCAM, are likely to provide an incomplete picture of metastasis. Accordingly, improved markers and techniques for detection of circulating epithelial cells (CECs), including CTCs, are needed for early diagnosis and prognosis of cancer and pre-cancer.

### SUMMARY

The present disclosure is based, at least in part, on the discovery that the release of epithelial cells into the bloodstream precedes clinically detectable tumor formation. Accordingly, the disclosure provides methods for identifying and characterizing the origin of circulating epithelial cells (CECs) (including circulating tumor cells (CTCs)), or fragments thereof, using tissue-specific markers as biomarkers, such as the pancreas-specific biomarker Pdx-1. In one embodiment, the identification of tissue-specific CECs in a biological fluid sample (e.g., 1, 2, 3, 4, 5, 10, 15, 20, 25, 30, or more CECs, or another defined minimum number depending on the patient), indicates an increased risk of dysplasia, or an increased risk for development of a tumor originating from that organ, as compared to a control.

In another embodiment, the identification of tissue-specific CTCs in a biological fluid sample indicates the presence of a tumor or metastases therefrom, in a subject. The number of CECs and CTCs in the blood also correlates with disease progression. Thus, the methods of the disclosure provide for the identification of patients who harbor dysplastic lesions that have a likelihood of progressing to cancer (e.g., dysplasia), the early prognosis and diagnosis of cancer (e.g., identification of small populations of cancer cells prior to identification of a tumor by conventional means), determination of stage of cancer progression, and therapy monitoring in a subject.

In one aspect, the present disclosure is directed to methods of assessing whether a subject is at risk for developing cancer, e.g., pancreatic cancer, comprising determining the presence of circulating epithelial cells in a biological fluid sample obtained from the subject, wherein presence of the circulating cells (CECs) is an indication that the patient is at increased risk for developing cancer. In one embodiment, the presence of CECs indicates dysplasia in the subject. In another embodiment, the method is carried out prior to the identification of a primary tumor in the subject. In another embodiment, the subject is at increased risk for developing pancreatic cancer.

In another aspect, the present disclosure is directed to methods for assessing whether a subject has cancer, e.g., pancreatic cancer, that has metastasized or is likely to metastasize, comprising determining the presence of circulating tumor cells in a biological fluid sample obtained from the subject, wherein presence of the circulating cells is an indication that the subject has cancer that has metastasized or is likely to metastasize.

In yet another aspect, the present disclosure provides methods of assessing the efficacy of a therapeutic or prophylactic therapy for preventing, inhibiting or treating cancer, e.g., pancreatic cancer, in a subject, comprising determining the level of circulating epithelial cells or circulating tumor cells in a biological fluid sample obtained from the subject prior to therapy; and determining the level of circulating epithelial cells or circulating tumor cells in a biological fluid sample obtained from the subject at one of more time points during therapeutic or prophylactic therapy, wherein the therapy is efficacious for preventing, inhibiting, or treating cancer in the subject when there is a lower level of circulating cells in the second or subsequent samples, relative to the first sample.

The methods of the disclosure can also be used for diagnosis or prognosis or assessment of disease-specific activity of non-cancer diseases or disorders, such as, but not limited to, pancreatitis or Inflammatory Bowel Disease (IBD). Accordingly, in one aspect the present disclosure provides methods of diagnosing or predicting the likelihood of developing an inflammatory disease in a subject by determining the presence of circulating epithelial cells in a biological fluid sample obtained from the subject, wherein presence of the circulating cells is an indication that the subject is afflicted with or likely to develop an inflammatory disease. In another embodiment, the present disclosure provides methods to monitor inflammatory disease activity, including response to treatment, after the diagnosis of this disease has already been made, during treatment of said disease. In one embodiment, the inflammatory disease is a gastrointestinal inflammatory disease.

In some embodiments, the presence of circulating cells in the biological fluid sample is determined using an immunocapture microfluidic device to capture the cells, wherein the microfluidic device contains antibodies specific for epithelial cells, e.g., antibodies to epithelial cell adhesion molecule (EpCAM). In another embodiment, the method further includes identifying the tissue of origin of the circulating cells by determining the presence of a tissue-specific biomarker expressed by the circulating cells, e.g., Pdx-1.

In one embodiment, the biomarker is a protein and the presence of the protein is detected using a reagent which specifically binds with the protein. For example, the reagent can be selected from the group consisting of an antibody, an antibody derivative, an antigen-binding antibody fragment, and a non-antibody peptide which specifically binds the protein. In another embodiment, the antibody or antigen-binding antibody fragment is a monoclonal antibody or antigen-binding fragment thereof, or a polyclonal antibody or antigen-binding fragment thereof.

The biomarker can also be a transcribed polynucleotide or portion thereof, e.g., a mRNA or a cDNA. In one embodiment, detecting a transcribed polynucleotide includes amplifying the transcribed polynucleotide.

The disclosure also provides kits for assessing whether a subject is afflicted with cancer or at risk for developing cancer, e.g., pancreatic cancer, or diagnosing or assessing the level of inflammation, containing reagents useful for detecting capturing CTCs or CECs and a tissue-specific biomarker.

### BRIEF DESCRIPTION OF THE FIGURES

The patent or application file contains at least one drawing executed in color. Copies of this patent or patent application publication with color drawing(s) will be provided by the Office upon request and payment of the necessary fee

FIGS. 1A-Q. Lineage-Labeled Mouse Models of Pancreatic Cancer and Detection of EMT. (A) Schematic of the PKCY mouse model used in this study, which employs the KrasG12D (“K”), Pdx1-Cre (“C”), p53 (“P”), and RosaYFP (“Y”) alleles. Cre-mediated activation of Kras and deletion of one allele of the p53 tumor suppressor are accompanied by recombination of the YFP lineage label. (B) Bright-field and fluorescent images of midgut organs from a CY mouse showing robust and specific fluorescence of the pancreas (outlined); some labeling is also present in the duodenum. (C) Time course of malignant progression in PKCY mice. (D-F) Representative images of malignant progression. Prior to weaning, PKCY mice have histologically normal pancreata (D) but develop PanIN lesions (E) and eventually PDAC (F). (G-I) Images of pancreata from (D)-(F) stained with an antibody against YFP and N-cadherin (N-cad); prior to weaning, scant N-cad staining is seen (G). (J and K) Fluorescent images of lineage-labeled cells derived from the pancreatic epithelium. In control (CY; Pdx-Cre; YFP) pancreata, YFP⁺ cells express E-cadherin (E-cad; J) but not N-cad (K). Dotted lines indicate YFP⁻ mesenchymal cells. (L)-(Q) Tumor cells were detected in tumor-bearing mice (“PDAC mice”) that coexpressed either Zeb1 or Fsp1 and the epithelial marker E-cadherin (E-cad) (O). The YFP lineage label was then used to identify PDAC cells that had completed an EMT. It was observed that 42% of the lineage-labeled YFP⁺ cells in PKCY tumors had undergone EMT (FIG. 1P); higher rates of EMT (68% of all YFP⁺ cells) were found in the IKCY model. EMT was not detected in lineage-labeled CY control mice by either method (FIG. 1L-1N).

FIGS. 2A-L. Detection of EMT in PKCY Mice using Additional Markers (related to FIG. 1). (A-D) Detection of EMT using lineage labeling and staining for the mesenchymal marker Fsp1. Similar to experiments with Zeb1 (FIG. 3), PKCY mice at the PanIN stage exhibit EMT (arrows) in PanIN 2 (B), and PanIN 3 (C) but not in PanIN 1 lesions (A). Numerous YFP⁺Fsp1⁺ cells are found concentrated at the tumor edge in PDAC mice (D). A goat anti-GFP antibody was used to visualize YFP (denoted by “YFP (g)”). (E and F) The mesenchymal marker Slug is expressed in the islets of control (Pdx1-Cre; RosaYFP) mice (E, inset) and in PanIN lesions of 10-week-old PKCY mice (F, inset). (G-J) Snail1 and SIP1 expression is not detected in control pancreata (G and I), but both proteins are expressed in a mosaic fashion within PanIN lesions in 10-week-old PKCY mice (H and J). For SIP1 staining, YFP was stained with a chicken anti-GFP antibody (denoted by “YFP(c)”), accounting for the different quality of staining. (K and L) E-cad expression is detected in acini and ducts in both control and PanIN mice (K, L). All cells within PanIN lesions exhibited strong membrane expression (L), indicating that acquisition of mesenchymal markers precedes E-cad loss during EMT.

FIGS. 3A-G. EMT Precedes Tumor Formation. (A-D) In pancreata taken from 8- to 10-week-old PKCY mice, EMT is observed in regions of acinar-to-ductal metaplasia with inflammation (ADMI; D), PanIN 2 (B), PanIN 3 (C), but not in PanIN 1 lesions (A). Arrows show individual YFP⁺ cells that also express Zeb1. (E) Quantification of observations from (A)-(D), showing the percentage of each type of lesion having at least one cell that has undergone EMT; numbers reflect at least ten medium-powered fields from each of five PanIN mice. (F) Strategy for isolating YFP+ epithelially derived cells from the pancreas; the purity of the YFP⁺ population was confirmed by a repeat FACS analysis. (G) Transcriptional analysis of sorted YFP⁺ pancreas cells from lineage-labeled CY control (n=4), PanIN (n=6), and PDAC (n=5) pancreata. Bar graph data are presented as mean±standard deviation (SD) in this and subsequent figures. *p<0.01; **p<0.001 bp two-tailed Student's t test in this and subsequent figures, unless otherwise noted. Scale bars, 20 mm. See also FIG. 4.

FIGS. 4A-B. EMT, Delamination, and Dissemination Occur within PanIN Pancreases and Are Concentrated near Regions of ADMIs (related to FIG. 3). (A) Three individual PKCY mice aged 8-10 weeks analyzed by IF (first column), H&E (second column), and FACS to determine CPC concentration (last column). IF staining for the YFP lineage label and Zeb1 or Fsp1 (insets) reveal EMT within all PanIN pancreata (arrows). YFP⁺Zeb1⁺ cells are concentrated in or near ADMIs (labeled). YFP⁺ cells that have delaminated from epithelial structures within the stroma assume a fibroblast-like appearance; most are Zeb1⁺ (arrows) and are indistinguishable from other stromal cells by H&E staining. Fsp1⁺ cells shown in the inset are distinct from the Zeb1⁺ cells denoted with arrows in the main panels. YFP was stained with Goat a GFP antibody (Abeam; YFP(g)). (B) Confirmation of pancreatic epithelial lineage of delaminated YFP⁺ cells using LSL-KrasG12D; Mist1-CreER; Rosa26LSL-YFP mice. Cre-recombinase expression in Mist1-expressing cells was elicited at 6 weeks of age using a 3 day pulse of tamoxifen. One week after treatment, chronic pancreatitis was induced using cerulein and mice were analyzed after 3 weeks. YFP⁺E-cad⁻ spindle-shaped cells were found to be dissociated from epithelial structures (arrowhead, inset) and were indistinguishable from surrounding stromal cells (H&E). YFP was stained with rabbit a GFP antibody (Invitrogen; “YFP(r)”).

FIGS. 5A-N. Hematogenous Spread and Liver Seeding Precede Tumor Formation. (A and B) Images showing individual YFP+ cells intermingled with stromal cells prior to tumor formation in a 10-week-old PKCY PanIN mouse (A). Delaminated YFP⁺ cells have a spindle-shaped morphology and express Zeb1 (boxes i-iii); they are indistinguishable from surrounding Zeb1⁺YFP⁻ stromal cells by H&E staining of an adjacent section (B). (C and D) FACS analysis of blood samples from age-matched CY control (C) and PKCY PanIN mice (D). YFP fluorescence and a stain for the leukocyte marker CD45 are depicted on the x and y axes of the FACS plot. YFP⁺CD45⁻ cells were seen in the blood of PanIN (D) and PDAC animals (boxed area indicates representative gating and absolute number of YFP⁺ cells). (E) Quantification of circulating YFP+ pancreatic cells (CPCs). Mean CPC numbers (per ml blood) were 3.65±3.76 (CY control, n=13), 32.8±26.2 (PanIN, n=17), and 97.3±48.9 (PDAC, n=18) (p<0.001). (F and G) Phase-fluorescent images showing epifluorescence of a sorted YFP+ cell. (H) Genomic PCR showing the presence of the recombined YFP allele in YFP+ cells but not YFP_ cells. Pancreatic DNA containing the recombined allele was included as a positive control. (I) Expression of transcripts encoding YFP, Pdx1, and E-cad, comparing sorted YFP+ and YFP_ cells and measured by qPCR (±SD). (J and K) Sanger sequencing after PCR amplification of cDNA showing that YFP+ CPCs express a mutant Kras allele that harbors an altered codon 12 (G/A, highlighted). (L-N) CPCs from 8- to 10-week-old PKCY animals seed the liver. (L) Micrometastasis in a liver from a tumor-bearing mouse (“PDAC Liver”). (M and N) Individual CPCs seed the liver at the PanIN stage (“PanIN Liver”); vascular lumens are outlined. Scale bar, 40 mm for (A and (B); 5 mm for (L)-(N). See also FIG. 6.

FIGS. 6A-D. Evidence of Delaminated Cells in Human PanIN Pancreata. (A) Pdx1 is expressed in a subset of delaminated YFP+ cells in PanIN mice (inset). (B-D) Immunohistochemical staining for Pdx1 in sections of human pancreata with PanIN lesions, demonstrating delaminated Pdx1+ cells from three different patients. Pancreatic specimens were obtained from patients who had undergone resection for PDAC; however, the examined portions of the pancreas in these sections were cancer free.

FIGS. 7A-F. CPC Characterization. (A) Quantification of CPCs after sampling from the left atrium or ventricle (“Left”) or right atrium or ventricle (“Right”) of the same animal (n=3). (B) Quantification of FACS staining for epithelial and mesenchymal markers in CPCs obtained from PanIN or PDAC mice (n=6-8 for each data point). (C and D) Quantification of YFP⁺ cells from the pancreas (C) and circulation (D) in PKCY PanIN and PDAC mice that stained positive for the putative pancreatic cancer stem cell markers CD24 and CD44. (E and F) Quantification of survival (E) or clonal growth (F) of YFP⁺ cells obtained from lineage-labeled control (CY), PanIN, and PDAC mice in ultra-low attachment wells. Bar graphs show the number of wells (out of 96 wells seeded with a single cell) exhibiting any live YFP⁺ cells (E, inset) or evidence of clonal growth (F, inset) after 7 days. p values for paired two-tailed Student's t tests are shown. Scale bars, 10 mm. See also FIG. 8.

FIGS. 8A-C. Expression of EpCAM and Zeb1 in CTCs and Characterization of CPCs from Cerulein-Treated KCY Mice. (A) Representative two-dimensional analysis of CTC from PKCY mice (n=3). Enriched blood specimens from PDAC mice were stained for the mesenchymal marker Zeb1 (x axis) and the epithelial marker EpCAM (y axis). The gated YFP+ CTC population is shown. Only 18.2% of all YFP⁺ CTC are double positive, suggesting that few CTC are in a “transitional” EMT state. (B) Quantification of YFP⁺ cells from the pancreas and circulation (CPC) in KCY mice treated with cerulein that stained positive for the putative pancreatic cancer stem cell markers CD24 and CD44. p value for a paired two-tailed Student's t test is shown. (C) Quantification of clonal growth of YFP⁺ pancreas and circulating (CPC) cells obtained from cerulein-treated KCY mice in ultra-low attachment wells. Bar graphs show the number of wells (out of 96 wells seeded with a single cell) exhibiting evidence of clonal growth after 7 days.

FIGS. 9A-I. Epithelial and Mesenchymal States Are Plastic. (A) Schematic of orthotopic transplantation experiments. (B-E) Fluorescent images taken 3 weeks after transplantation of YFP⁺ cells from PDAC mice into NOD/SCID hosts. Tumors form in all mice regardless of E-cad status (n=5 for each condition). YFP⁺E-cad⁺ and YFP⁺E-cad⁻ cells are present in both conditions (C and E), as are YFP⁺Zeb1⁺ and YFP⁺Zeb1⁻ cells (B and D). (F-I) Fluorescent images taken 8 weeks after transplantation of YFP⁺ cells from PanIN mice into NOD/SCID hosts. After transplantation of YFP⁺E-cad⁺ cells, no tumors are found (n=6); the few transplanted YFP⁺ cells that remain are Zeb1⁻ and E-cad⁺ (F and G). Transplantation of YFP⁺E-cad⁻ cells results in tumor formation (H and I). Tumors contain both E-cad and E-cad⁻ cells (I) as well as Zeb1⁺ and Zeb1⁻ cells (H), providing direct evidence for MET.

FIGS. 10A-N. Inflammation Augments EMT and Dissemination. (A-F) Fluorescent images of 8-week-old control Pdx1-Cre; Rosa YFP (CY; A and D), KrasG12D; Pdx1-Cre; Rosa YFP (KCY; B and E), and PanIN (C and F) pancreata 3 days after injection with vehicle (A-C) or cerulein (D-F) to induce acute pancreatitis (n=3 for each condition). YFP⁺Zeb1⁺ cells present in PanIN mice or observed following cerulein treatment of CY and KCY mice are shown (C-F, insets). (G) Quantification of CPC number after 3 days of treatment with vehicle or cerulein for mice with the indicated genotypes (n=3 for each group; p<0.05 comparing the two groups of KCY mice by Student's t test; p=0.014 for the effect of cerulein versus control across all groups by two-way ANOVA). (H-M) Images of 10-week-old control (H and K) and PanIN pancreata (I and J, L and M) after 7 days of treatment with vehicle (DMSO; H-J) or dexamethasone (Dex; K-M) and analyzed 24 hr after the last injection. YFP⁺Zeb1⁺ cells are seen in vehicle-treated controls (J, inset) but not in Dex-treated animals (M, inset). (N) Quantification of CPC number in vehicle- and Dex-treated PanIN mice (p=0.029, n=6 for each group). See also FIGS. 11 and 12.

FIGS. 11A-F. Effect of Acute Experimental Pancreatitis Elicited by Cerulein on EMT. (A-D) Cerulein treatment of control (A and B) and KCY (C and D) mice leads to the emergence of YFP⁺Zeb1⁺ cells (insets). Note the accumulation of YFP⁻Zeb1⁺ stromal cells in both conditions. There is little change in the frequency of EMT that is found when PDAC mice are treated with cerulein (F) compared to vehicle (E). Dashes denotes approximate boundary between tumor (to the right and below) and macroscopically unaffected pancreas.

FIGS. 12A-N. Effect of Chronic Pancreatitis Elicited by Main PDL and Effect of Cerulein, Dexamethasone, and TGF-b on PKCY Cells In Vitro. (A-F) PanIN mice were randomized to undergo PDL (D-F, n=4) or sham laparotomy (A-C, n=3). One week after surgery, pancreata from PDL mice were larger and more nodular (D) than sham treated mice (A) and mice with no laparotomy (data not shown). Pancreata from PDL-treated mice distal to the ligation (E and F) contained significant inflammation and more PanIN 3 lesions compared to Sham-treated pancreata (B and C; p<0.05). The pancreas distal to ligation also lacked any discernible acinar structures. (G) Quantification of CPCs in Sham-treated and PDL-treated PanIN mice. Asterisk denotes a mouse in which PDL surgery resulted in no significant inflammation by H&E analysis (i.e., the surgery was likely a technical failure). This value was included in the unpaired two-tailed Student's t test noted on the graph. (H-N) Effect of cerulein, dexamethasone and TGF-b on PKCY cells in vitro. Cell lines established from YFP⁺ sorted cells from PanIN mice were exposed to cerulein, dexamethasone, TGF-b, or DMSO vehicle. After 48 hr, cells treated with cerulein (100 nM; I) and dexamethasone (Dex, 1 mM; J) had identical morphology as vehicle-treated cells (H). However, TGF-b (5 ng/ml) exposure resulted in the acquisition of a fibroblast-like morphology (K) which was not rescued by adding Dex to the media (L). Cerulein and Dex treatment had no effect on proliferation (M) or the expression of a panel of epithelial or mesenchymal markers (N), though TGF-b exposure resulted in significant downregulation of epithelial markers and an increase in the expression of the mesenchymal markers Zeb1 and N-cad. Studies were performed in triplicate for each condition. *p<0.01; **p<0.001 bp two-sided Student's t test.

FIG. 13. Human pancreatic cancer cells in blood express Pdx-1. Human pancreatic cancer cells were isolated in blood and nucleic acids released afterwards. Quantitative PCR for Pdx-1 reveals that 5/5 specimens with cancer cells and 0/3 specimens from non-cancer controls contained Pdx-1.

FIG. 14A-B. Exemplary mRNA (A) (SEQ ID NO:1) and protein (B) (SEQ ID NO:2) sequences for Pdx-1.

FIG. 15. Geometrically Enhanced Differential Immunocapture (GEDI). GEDI, an exemplary method for capture of CECs, is depicted.

FIGS. 16A-D. Detection of CECs in patients using GEDI. (A) Depiction of the GEDI device. (B) Vertical scatterplots of CEC concentrations (per ml blood) for cancer-free patients (Control), patients with cystic lesions of the pancreas without dysplasia or tumor (cystic lesion) and patients with PDAC. Lines indicate means. Bars indicate statistically significant differences by Mann-Whitney test. Representative images of individual GEDI-captured nucleated cells from (C) control human blood spiked with PI34 cells and (D) blood from a patient with PDAC. Cells were stained for CD45, Pdx-1 and DNA (DAPI). Scale bars in C and D represent 20 μm.

### DETAILED DESCRIPTION

The present disclosure is based, at least in part, on the discovery that epithelia-to-mesenchymal transition (EMT), migration of epithelially derived cells into the stroma, bloodstream entry of epithelial cells, and seeding of the liver occur at a stage of pancreatic adenocarcinoma progression previously thought to be preinvasive based on standard histological examination. Thus, this disclosure provides a model for cancer progression, e.g., pancreatic cancer progression, in which the seeding of distant organs occurs before, and in parallel to, tumor formation at the primary site. Furthermore, the present disclosure is based, at least in part, on the discovery made in clinical studies, and described herein, that circulating epithelial cells (CECs) are indicative of the presence of dysplastic lesions in the pancreas, prior to tumor formation and prior to the histologic appearance and clinical diagnosis of cancer.

Accordingly, based on the discovery that EMT and epithelial cell dissemination into the bloodstream precede tumor formation, and the discovery that CECs are indicative of the presence of dysplasia on histology, the disclosure provides methods for identifying and characterizing the origin of circulating epithelial cells (CECs) using tissue-specific markers, and using these cells as biomarkers to assess risk for cancer development in the patient prior to identification of a tumor in the patient, wherein the presence of CECs in a biological fluid sample (e.g., 1, 2, 3, 4, 5, 10, 15, 20, 25, 30, or more), or another defined minimum number depending on the patient, indicates an increased risk for the development of cancer in the patient.

Epithelial cells do not normally enter the bloodstream in adults. Therefore, the identification of tissue-specific CECs in the blood or other biological fluid indicates an increased risk for development of a tumor originating from that organ or tissue (e.g., the presence of dysplasia or a pre-cancerous condition). Thus, CECs are biomarkers for dysplasia, or abnormal epithelial cells, that can progress to cancer. The identification of tissue-specific CTCs in the blood or other biological fluid indicates the presence of a tumor or metastases therefrom, in a subject.

Current medical practice relies on invasive techniques for identifying dysplasia, such as, for example, needle biopsy or removal of polyps via colonoscopy, or enhanced imaging techniques. However, these methods are imperfect due to their invasive nature and/or the inaccessibility of certain organs which makes biopsy or imaging difficult or impossible. Detection of CECs in a biological fluid, as described herein, provides a non-invasive, specific and sensitive biomarker for dysplasia from any epithelial organ.

Furthermore, the number of CECs and CTCs in the blood or other biological fluid also correlates with disease progression. Thus, the methods of the disclosure provide for early prognosis and diagnosis of cancer and therapy monitoring in a subject.

Based on the identification and characterization of circulating cells, e.g., to identify the tissue or organ of origin, a diagnosis of cancer, or prediction of assessment of risk for cancer (e.g., dysplasia), in any epithelial organ in the subject can be made, even prior to the development, or identification of, tumor formation at the primary site, thus allowing for prophylactic therapy in certain subjects. For example, further or more frequent monitoring, biopsy, surgical resection, or other prophylactic measures to prevent tumor formation or identify cancer at a very early stage can be carried out based on the detection of CECs from any particular organ in a biological sample.

In addition, a determination of the level of risk for cancer progression or the stage of cancer progression can be made based on the number of CECs or CTCs in circulation. For example, as described in Example 2, on average, the number of CECs in patients with pancreatic ductal adenocarcinoma (PDAC) was significantly higher than the number of CECs detected in patients with precancerous cystic lesions and not diagnosed with cancer, indicating that the greater the number of CECs present in a biological fluid sample, the higher the risk for developing cancer or the presence of a tumor (see Table 3).

Furthermore, the effectiveness of cancer therapy can be monitored by evaluating the presence and number of CECs and CTCs in circulation over the course of therapy, and decisions can be made regarding the type, duration, and course of therapy based on these evaluations.

In one embodiment, the subject being tested for the presence of CECs in a biological sample, as described herein, can be a subject who is at high risk for developing cancer. In one embodiment, a subject is at high risk for development of cancer based on, for example, family history or determination of genetic predisposition. For example, these findings have implications for the management of individuals at high risk for cancer, e.g., pancreatic cancer, including subjects with hereditary or chronic pancreatitis or kindreds with inherited pancreatic cancer. Based on the discovery that dissemination and seeding of epithelial cells precede the detection of a tumor or the histologic appearance or diagnosis of cancer, a window of opportunity exists for prophylactic therapy in high-risk subjects in the time-period prior to detection of a tumor.

In one embodiment, and as exemplified herein, the pancreas-specific marker Pdx-1 (“pancreatic and duodenal homeobox 1”) can be used to characterize CECs or CTCs as cells of pancreatic origin. The release of Pdx-1 positive cells into the bloodstream precedes the pancreas tumor formation and its number also correlates with the disease progression. By analyzing Pdx-1 levels on the CECs or CTCs (or circulating rare cells) isolated by any known cell capture or enrichment method, adverse pancreatic lesion events can be identified more precisely and at a much earlier stage.

As exemplified herein, the present disclosure can be used to diagnose pancreatic ductal adenocarcinoma (PDAC), as the vast majority of patients with pancreatic cancer have metastatic disease at the time of diagnosis using current methods. More than 75% of patients who undergo surgical resection of small pancreatic tumors with clear surgical margins and no evidence of metastasis die from metastatic disease within 5 years (Neoptolemos et al., 2004), a finding that is consistent with early spread. Moreover, metastatic PDAC has been documented in a cohort of patients who underwent pancreatectomy for chronic pancreatitis and in whom histologic analysis of the resected pancreas revealed only PanIN lesions (Sakorafas and Sarr, 2003). Accordingly, diagnosis and treatment at a very early stage is essential.

The methods of this disclosure are not limited to the use of Pdx-1 as the biomarker, as any tissue-specific biomarker can be utilized. For example, HNF4α (Hepatocyte nuclear factor 4 alpha; Chartier F L, et al. B (September 1994). Gene 147 (2): 269-72) can be used as a biomarker for liver cancer; and testes specific protein TPX1 (also known as CRISP2, cyctein-rich secretory protein 2; Busso D, et al. (2005). Mol. Hum. Reprod. 11 (4): 299-305) can be used as a biomarker for testicular cancer. Therefore, the methods of the present disclosure are not limited to diagnosis and prognosis of pancreatic cancer, but are applicable to any cancer of epithelial origin, including, for example, liver, testicular, breast, colon, prostate, or lung cancer.

Furthermore, the methods of this disclosure can also be used to diagnose or predict other, non-cancer diseases or disorders. As described herein, epithelial cells contained in gastrointestinal (GI) organs, and not normally in the blood stream, are released into the blood circulation during inflammation and become CECs. These CECs correlate with the histological degree of inflammation in the solid organs. Therefore, CECs from biological fluids (for example, blood), can be used as a biomarker for the presence and degree of inflammatory disease in the GI tract, or other organs. This method therefore represents a quantitative, non-invasive and cost-effective method of diagnosis and monitoring of inflammatory diseases. For example, identification of circulating pancreas cells in the bloodstream can indicate pancreatitis. Identification of the presence of circulating colonic cells using a colon specific biomarker can indicate the presence of or risk for Inflammatory Bowel Diseases, such as ulcerative colitis and Crohn's disease. Other diseases or disorders that can be diagnosed and assessed by identifying the presence and/or number of CECs include, for example, chronic inflammatory diseases of the GI tract such as esophagitis, gastritis, and hepatitis. However, the methods of the present disclosure are not limited to these GI diseases but can be applied to inflammatory conditions afflicting other organs as well.

In one embodiment, the disclosure provides methods for detecting inflammation in organs to diagnose inflammatory disease by identifying CECs in a biological fluid. In another embodiment, since many inflammatory diseases are not curable and require long-term treatment, the methods of the disclosure can be used to monitor the efficacy of treatment of an inflammatory disease over time by monitoring and evaluating the presence of CECs in a biological fluid at multiple time points throughout the course of therapy. Determinations regarding the type, frequency, duration, and course of therapy can be made based on these evaluations.

### DEFINITIONS

“Circulating epithelial cells” or “CECs” are epithelial cells that have detached from a primary organ and are circulating in the bloodstream or other bodily fluid including, but not limited to, lymphatic fluid and ascites. As provided herein, circulating epithelial cells have been found in the bloodstream in the absence of cancers of epithelial origin and hence are not necessarily associated with cancer, but are indicative of dysplasia or inflammation.

“Circulating tumor cells” or “CTCs” are epithelial cancer cells that have detached from a tumor and are circulating in the bloodstream or other bodily fluid including, but not limited to, lymphatic fluid and ascites.

“Cancers of epithelial origin” refers to “carcinoma” that arise from epithelial cells which include, but are not limited to, pancreatic cancer, skin cancer, lung cancer, breast cancer, prostate cancer, renal cell carcinoma, liver cancer, urinary bladder cancer, ovarian cancer, cervical cancer, endometrial cancer, gastrointestinal cancers including esophageal cancer, small bowel cancer and stomach cancer, colon cancer, and other known cancers that effect epithelial cells throughout the body. A patient carries the diagnosis of carcinoma when histologic or cytologic analysis reveals the presence of cells that appear to have features of cancer cells, such as invasion through basement membrane and extreme abnormalities in the shape of the cell or nucleus.

As used herein, the term “dysplasia” refers to the histologic appearance of abnormal epithelial tissue, e.g., alteration in size, shape, and/or organization of cells, that can progress to cancer. Dysplasia is often a precursor to tumor formation, but is a pre-cancerous state. Dysplasia is a histologic or cytologic definition, regarding the appearance of cells upon examination of a tissue specimen. There can be different grades of dysplasia, according to how abnormal these cells appear upon examination. Importantly, clinical analysis of tissue and cytologic specimens for cells with dysplasia is not always predictive of the presence of such cells in the patient. For example, the absence of cells with dysplasia on biopsy does not always mean that there are no cells with dysplasia in the patient from which the biopsy was taken.

The term “dysplastic cells” refers to cells displaying the histologic appearance of abnormal tissue that can progress to cancer.

The term “metastasis,” as used herein, refers to the condition of spread of cancer from the organ of origin to additional distal sites in the subject.

A “biological sample” refers to a sample of biological material obtained from a subject, preferably a human subject, including a tissue, a tissue sample, a cell sample, a tumor sample, and a biological fluid, e.g., blood, urine, lymphatic fluid, ascites, and a nipple aspirate. In one non-limiting embodiment, CECs and CTCs are captured from a peripheral blood sample obtained from a subject.

A “primary tumor” is a tumor appearing at a first site within the subject and can be distinguished from a “metastatic tumor” which appears in the body of the subject at a remote site from the primary tumor.

A “patient” or “subject,” as used interchangeably herein, refers to any warm-blooded animal, preferably a human.

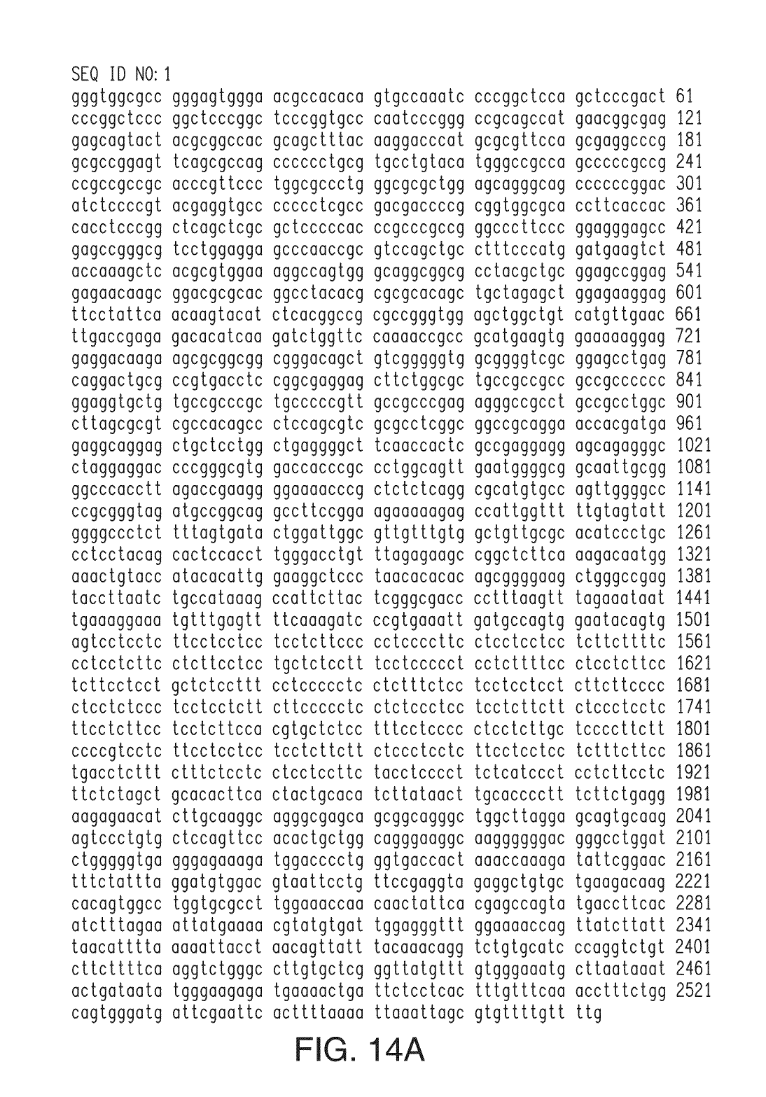

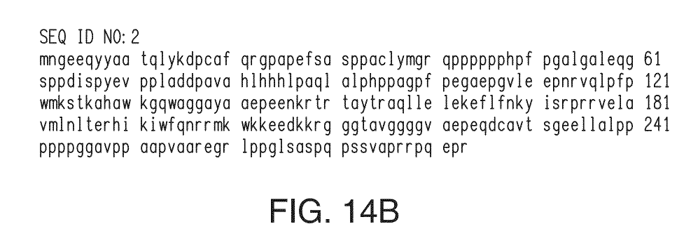

“Pdx-1” or “pancreatic and duodenal homeobox 1” is a transcriptional activator of several genes, including insulin, somatostatin, glucokinase, islet amyloid polypeptide, and glucose transporter type 2. The encoded nuclear protein is involved in the early development of the pancreas and plays a major role in glucose-dependent regulation of insulin gene expression. An exemplary nucleotide sequence of human Pdx-1 is provided in Genbank Accession No.: Chromosome: 13, NC_{—}000013.10 (28494168.28500451), GI:224589804 (genomic sequence); and NM_{—}000209.3, GI:189095257 (mRNA sequence) (SEQ ID NO:1; FIG. 14A). An exemplary amino acid sequence of human Pdx-1 is provided in GenBank Accession No. P52945.1; GI:1708540 (SEQ ID NO:2; FIG. 14B).

A “tissue-specific biomarker” can be any nucleic acid or protein marker that is sufficiently specific for a particular tissue type and allows for the reasonable identification of the organ of origin of a particular circulating epithelial or tumor cell.

### Prognostic and Diagnostic Methods

The methods for detecting the presence of CECs or CTCs in a biological fluid and determining the tissue of origin using tissue-specific biomarkers, e.g., Pdx-1 on pancreatic cells, have many applications. For example, one or more tissue-specific markers can be identified on CECs to aid in cancer prognosis, e.g., at an early stage, for example, in determining the presence of dysplasia prior to tumor formation or detection by conventional means, or for predicting, diagnosing or monitoring an inflammatory disease. In another example, the methods for detection of the circulating cells can be used to monitor responses in a subject to prophylactic or therapeutic treatment (for example, preventative cancer treatment, treatment of diagnosed cancer, or treatment of inflammation). In another embodiment, the circulating cells are used to predict the level of risk of a subject developing cancer, or differentiate between the different stages of tumor progression, thus aiding in determining appropriate treatment and extent of metastasis of the tumor.

The methods described herein are useful for predicting subjects at risk for developing cancer or diagnosing subjects who have any one of a variety of epithelial cancers. For example, the cancer can be pancreatic cancer, kidney cancer, e.g., renal cell carcinoma (RCC), urogenital cancer, e.g., urothelial carcinomas in urinary bladder, kidney, pelvic and ureter, melanoma, prostate carcinoma, lung carcinomas (non-small cell carcinoma, small cell carcinoma, neuroendocrine carcinoma and carcinoid tumor), breast carcinomas (ductal carcinoma, lobular carcinoma and mixed ductal and lobular carcinoma), thyroid carcinomas (papillary thyroid carcinoma, follicular carcinoma and medullary carcinoma), brain cancers (meningioma, astrocytoma, glioblastoma, cerebellum tumors, medulloblastoma, ependymoma), ovarian carcinomas (serous, mucinous and endometrioid types), cervical cancers (squamous cell carcinoma in situ, invasive squamous cell carcinoma and endocervical adenocarcinoma), uterine endometrial carcinoma (endometrioid, serous and mucinous types), primary peritoneal carcinoma, mesothelioma (pleura and peritoneum), eye cancer (retinoblastoma), muscle (rhapdosarcoma and leiomyosarcoma), lymphomas, esophageal cancer (adenocarcinoma and squamous cell carcinoma), gastric cancers (gastric adenocarcinoma and gastrointestinal stroma tumor), liver cancers (hepatocellular carcinoma and bile duct cancer), small intestinal tumors (small intestinal stromal tumor and carcinoid tumor) colon cancer (adenocarcinoma of the colon, colon high grade dysplasia and colon carcinoid tumor), testicular cancer, skin cancers (melanoma and squamous cell carcinoma) and adrenal carcinoma.

Furthermore, the methods of this disclosure can also be used to predict, assess, diagnose, or monitor, other, non-cancer diseases or disorders. For example, pancreatitis or diseases or disorders which result in circulating colon cells, e.g., inflammatory GI diseases including Inflammatory Bowel Disease, such as ulcerative colitis, or other inflammatory diseases affecting other organs.

Additionally, disease progression can be assessed by following expression of tissue-specific markers, e.g., Pdx-1 in CECs or CTCs in individual patients over time. Accordingly, methods provided herein also include methods for monitoring the progression of cancer in a subject, comprising, e.g., monitoring the presence or number of CECs or CTCs expressing a tissue-specific biomarker over time. An increase in the percentage of CECs or CTCs expressing the tissue-specific biomarker, e.g., Pdx-1, over time, indicates that the cancer is progressing or has developed. In another embodiment, a reference reading is taken after surgical removal of tissue, e.g., cancerous tissue, then another taken at regular intervals. CEC detection can also be used to monitor recurrence of cancer during remission, wherein frequent samples are analyzed to confirm that the patient is free of dysplasia and cancer.

In another embodiment, methods provided herein also include methods for monitoring the progression of an inflammatory disease. For example, an increase in the percentage of CECs expressing a tissue-specific marker indicates an increase in inflammation in the tissue. CEC detection can be done over the course of therapy to aid the physician in adjusting anti-inflammatory medications, for example in comparison to a reference reading taken at an initial time point or prior to treatment. CEC detection can also be used as a periodic assessment of disease activity during stages of remission.

The information provided by the methods described herein can be used by the physician in determining the most effective course of treatment (e.g, preventative or therapeutic treatment). A course of treatment refers to the prophylactic measures taken for a patient after the assessment of increased risk for development of cancer is made. A course of treatment can also refer to treatment given after the diagnosis of cancer or an inflammatory disease is made. For example, a determination of an increased likelihood of cancer occurrence in a subject based on the identification of CECs (in the case of dysplasia, prior to tumor formation or identification), recurrence, spread, or patient survival, can assist in determining whether a more conservative or more radical approach to therapy or prophylaxis should be taken, or whether treatment modalities should be combined. For example, when CECs are identified the subject is identified as having an increased risk for developing cancer. At that stage, a physician can determine whether prophylactic measures, such as change in diet, further or more frequent monitoring, biopsy, surgical resection, or other prophylactic measures should be undertaken. Also, when cancer progression or metastasis is determined to be likely (e.g., based on a large number of CECs or identification of CTCs), it can be advantageous to precede or follow surgical treatment with chemotherapy, radiation, immunotherapy, biological modifier therapy, gene therapy, vaccines, and the like, or adjust the span of time during which the patient is treated.

Determining the presence of CECs or CTCs expressing a tissue-specific biomarker, e.g., Pdx-1, can also be combined with the detection of one or more other biomarkers for which increased or decreased expression correlates with cancer. The selected biomarker can be a general diagnostic or prognostic marker useful for multiple types of cancer, or can be a cancer-specific diagnostic or prognostic marker, such as a colon cancer marker (for example, sialosyl-TnCEA, CA19-9, or LASA), breast cancer marker (for example, CA 15-2. Her-2/neu and CA 27.29), ovarian cancer marker (for example, CA72-4), lung cancer (for example, neuron-specific enolase (NSE) and tissue polypeptide antigen (TPA)), prostate cancer (for example, PSA, prostate-specific membrane antigen and prostatic acid phosphatase), melanoma (for example, S-100 and TA-90), as well as other biomarkers specific for other types of cancer. Those skilled in the art will be able to select useful diagnostic or prognostic markers for detection in combination with CECs or CTCs expressing a tissue-specific biomarker, e.g., Pdx-1 or other tissue-specific biomarkers of the present disclosure or known in the art. Similarly, three or more, four or more or five or more or a multitude of biomarkers can be used together for determining a diagnosis or prognosis of a subject.

Also provided herein are kits, e.g., kits for determining the presence of CECs or CTCs expressing a tissue-specific biomarker, e.g., Pdx-1, in a subject or in a biological sample of a subject. A kit can include a method for capturing circulating cells and/or any agent useful for detecting biomarker, e.g., Pdx-1, proteins or mRNA (including potentially pre-mRNA), such as agents further described herein. A kit can further include a control, such as a control value or control sample or control tissue. A control can be protein or RNA attached to a solid support. A kit can also include additional components or reagents necessary for the detection of a biomarker, e.g., Pdx-1, such as secondary antibodies for use in immunohistochemistry. A kit can further include one or more other biomarkers or reagents for evaluating other prognostic factors, e.g., tumor stage.

### Capture of Circulating Cells

Circulating endothelial or tumor cells can be isolated from a subject by any means known in the art or described herein. Circulating cells can be isolated from a biological sample obtained from a subject, such as a whole blood sample, or other biological fluid.

There are several capture and enrichment platforms that are known in the art and currently available. For example, cells can be captured using antibody based systems. An example of non-microfluidic antibody based methods are the CellSearch™ systems available from Veridex, LLC™. The systems utilize immunomagnetic enrichment of EpCAM cells and three color fluorescence identification of DAPI+, cytokeratin 8/18/19+ and CD45− cells. The CellSearch™ Circulating Endothelial Cell Kit immunomagnetically captures CD 146-positive cells from whole blood.

As described herein, one technology that can be used to detect CECs is the Geometrically Enhanced Differential Immunocapture (GEDI) device. The GEDI platform is generally described in Gleghorn et al., 2009, the contents of which are expressly incorporated herein by reference. The GEDI device has been optimized for use in capture of epithelial cells by utilizing antibodies specific for an epithelial cell specific antigen, such as epithelial cell adhesion molecule (EpCAM). Following capture of CECs, a secondary screening method can be utilized to confirm the tissue of origin of the CECs, including, for example, screening for one or more tissue-specific biomarkers and/or additional epithelial markers or markers to identify nucleated cells, or other cell-type distinguishing markers.

Microfluidic platform based technology Cell Enrichment and Extraction (CEE), such as the OncoCEE Microchannel Chip,™ available from Biocept,™ can also be used. Another example of an antibody-based system is one where CTCs are enriched by integrating an antibody-coated silicon nanopillar (SiNP) substrate with an overlaid polydimethylsiloxane (PDMS) microfluidic chaotic mixer, as described in Wang et al, Angewandte Chemie International Edition Volume 50, Issue 13, pages 3084-3088, Mar. 21, 2011). Another example is the CTC Chip (Johnson and Johnson; Stott et al, Proc Natl Acad Sci USA 2010; 107: 18392-7; Nagrath et al, Nature 2007; 450:1235-9).

Circulating cells can also be captured and enriched using size-based systems. For example, ScreenCell™ provides filtration devices can be used to isolate and sort circulating rare cells by size, independent of EpCAM expression.

In addition, On-Q-ity™ CTC Chip employs an array of microfabricated posts coated with anti-EpCAM antibody to filter blood, and therefore combines size selectivity and microfluidic EpCAM capture. Apocell™ ApoStream™ technology isolates rare cells from whole blood without the use of EpCAM expression detection by exploiting morphological and electrical properties of the cells.

Other methods for capture of circulating cells include those described in, for example, U.S. Patent Application Publication Numbers US20120003711, US20110306043, US20110294186, US20110256155, US20110053152, US20080057505, US20020172987, and US20080206757, the contents of which are hereby incorporated by reference herein.

### Biomarker Detection

A tissue-specific biomarker used in the methods of the disclosure can be identified on CECs or CTCS using any method known in the art. Determining the presence of a tissue-specific marker, e.g., Pdx-1, protein or degradation product thereof, the presence of mRNA or pre-mRNA, or the presence of any biological molecule or product that is indicative of biomarker expression, or degradation product thereof, can be carried out for use in the methods of the disclosure by any method described herein or known in the art.

Protein Detection Techniques

Methods for the detection of protein biomarker are well known to those skilled in the art, and include ELISA (enzyme linked immunosorbent assay), RIA (radioimmunoassay), Western blotting, and immunohistochemistry. These methods use antibodies, or antibody equivalents, to detect protein. Antibody arrays or protein chips can also be employed, see for example U.S. Patent Application Nos: 20030013208A1; 20020155493A1, 20030017515 and U.S. Pat. Nos. 6,329,209; 6,365,418, herein incorporated by reference in their entirety.

ELISA and RIA procedures can be conducted such that a biomarker standard is labeled (with a radioisotope such as ¹²⁵I or ³⁵S, or an assayable enzyme, such as horseradish peroxidase or alkaline phosphatase), and, together with the unlabelled sample, brought into contact with the corresponding antibody, whereon a second antibody is used to bind the first, and radioactivity or the immobilized enzyme assayed (competitive assay). Alternatively, the biomarker in the sample is allowed to react with the corresponding immobilized antibody, radioisotope or enzyme-labeled anti-biomarker antibody is allowed to react with the system, and radioactivity or the enzyme assayed (ELISA-sandwich assay). Other conventional methods can also be employed as suitable.

The above techniques can be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods can also be employed as suitable.

In one embodiment, a method for measuring biomarker expression includes the steps of: contacting a biological specimen, e.g., circulating cells captured from a biological sample, e.g., blood, with an antibody or variant (e.g., fragment) thereof which selectively binds the biomarker, and detecting whether the antibody or variant thereof is bound to the sample. A method can further include contacting the specimen with a second antibody, e.g., a labeled antibody. The method can further include one or more steps of washing, e.g., to remove one or more reagents.

It can be desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene can provide a suitable support.

Enzymes employable for labeling are not particularly limited, but can be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase can be assayed by measuring the concentration of hydrogen peroxide formed after reaction of the enzyme-labeled antibody with the substrate under controlled conditions well-known in the art.

Other techniques can be used to detect a biomarker according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection can also be used.

Immunohistochemistry can be used to detect expression of a biomarker, e.g., in a biopsy sample. A suitable antibody is brought into contact with, for example, a thin layer of cells, washed, and then contacted with a second, labeled antibody. Labeling can be by fluorescent markers, enzymes, such as peroxidase, avidin, or radiolabelling. The assay is scored visually, using microscopy. The results can be quantitated.

Other machine or autoimaging systems can also be used to measure immunostaining results for the biomarker. As used herein, “quantitative” immunohistochemistry refers to an automated method of scanning and scoring samples that have undergone immunohistochemistry, to identify and quantitate the presence of a specified biomarker, such as an antigen or other protein. The score given to the sample is a numerical representation of the intensity of the immunohistochemical staining of the sample, and represents the amount of target biomarker present in the sample. As used herein, Optical Density (OD) is a numerical score that represents intensity of staining. As used herein, semi-quantitative immunohistochemistry refers to scoring of immunohistochemical results by human eye, where a trained operator ranks results numerically (e.g., as 1, 2 or 3).

Various automated sample processing, scanning and analysis systems suitable for use with immunohistochemistry are available in the art. Such systems can include automated staining (see, e.g, the Benchmark system, Ventana Medical Systems, Inc.) and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.).

Another method that can be used for detecting and quantitating biomarker protein levels is Western blotting. Cells can be frozen and homogenized in lysis buffer. Immunodetection can be performed with antibody to a biomarker using the enhanced chemiluminescence system (e.g., from PerkinElmer Life Sciences, Boston, Mass.). The membrane can then be stripped and re-blotted with a control antibody, e.g., anti-actin (A-2066) polyclonal antibody from Sigma (St. Louis, Mo.).

Antibodies against biomarkers can also be used for imaging purposes, for example, to detect the presence of a biomarker, e.g., Pdx-1, in cells of a subject. Suitable labels include radioisotopes, iodine (¹²⁵I, ¹²¹I), carbon (¹⁴C), sulphur (³⁵S), tritium (³H), indium (¹¹²In), and technetium (^{99m}Tc), fluorescent labels, such as fluorescein and rhodamine, and biotin. Immunoenzymatic interactions can be visualized using different enzymes such as peroxidase, alkaline phosphatase, or different chromogens such as DAB, AEC or Fast Red.

For in vivo imaging purposes, antibodies are not detectable, as such, from outside the body, and so must be labeled, or otherwise modified, to permit detection. Markers for this purpose can be any that do not substantially interfere with the antibody binding, but which allow external detection. Suitable markers can include those that can be detected by X-radiography, NMR or MRI. For X-radiographic techniques, suitable markers include any radioisotope that emits detectable radiation but that is not overtly harmful to the subject, such as barium or caesium, for example. Suitable markers for NMR and MRI generally include those with a detectable characteristic spin, such as deuterium, which can be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99 m.

The labeled antibody or antibody fragment will then preferentially accumulate at the location of cells which contain a biomarker, e.g., Pdx-1. The labeled antibody or variant thereof, e.g., antibody fragment, can then be detected using known techniques. Antibodies include any antibody, whether natural or synthetic, full length or a fragment thereof, monoclonal or polyclonal, that binds sufficiently strongly and specifically to the biomarker to be detected, e.g., human Pdx-1. An antibody can have a Kd of at most about 10⁻⁶M, 10⁻⁷M, 10⁻⁸M, 10⁻⁹M, 10⁻¹⁰M, 10⁻¹¹M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant.

Antibodies and derivatives thereof that can be used encompasses polyclonal or monoclonal antibodies, chimeric, human, humanized, primatized (CDR-grafted), veneered or single-chain antibodies, phase produced antibodies (e.g., from phage display libraries), as well as functional, i.e., Pdx-1 binding fragments, of antibodies. For example, antibody fragments capable of binding to a biomarker, e.g., Pdx-1 or portions thereof, including, but not limited to Fv, Fab, Fab′ and F(ab′)2 fragments can be used. Such fragments can be produced by enzymatic cleavage or by recombinant techniques. For example, papain or pepsin cleavage can generate Fab or F(ab′)2 fragments, respectively. Other proteases with the requisite substrate specificity can also be used to generate Fab or F(ab′)2 fragments. Antibodies can also be produced in a variety of truncated forms using antibody genes in which one or more stop codons have been introduced upstream of the natural stop site. For example, a chimeric gene encoding a F(ab′)2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies.

In some embodiments, agents that specifically bind to a polypeptide other than antibodies are used, such as peptides. Peptides that specifically bind can be identified by any means known in the art, e.g., peptide phage display libraries. Generally, an agent that is capable of detecting a biomarker, e.g., Pdx-1, polypeptide, such that the presence of a biomarker is detected and/or quantitated, can be used. As defined herein, an “agent” refers to a substance that is capable of identifying or detecting a biomarker such as Pdx-1 in a biological sample (e.g., identifies or detects Pdx-1 mRNA, Pdx-1 DNA, Pdx-1 protein). In one embodiment, the agent is a labeled or labelable antibody which specifically binds to a biomarker polypeptide.

In addition, a biomarker can be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modem laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait).

For additional information regarding mass spectrometers, see, e.g., Principles of Instrumental Analysis, 3rd edition. Skoog, Saunders College Publishing, Philadelphia, 1985; and Kirk-Othmer Encyclopedia of Chemical Technology, 4th ed. Vol. 15 (John Wiley & Sons, New York 1995), pp. 1071-1094.

Detection of the presence of a marker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

Any person skilled in the art understands, any of the components of a mass spectrometer (e.g., desorption source, mass analyzer, detect, etc.) and varied sample preparations can be combined with other suitable components or preparations described herein, or to those known in the art. For example, in some embodiments a control sample can contain heavy atoms (e.g., 13C) thereby permitting the test sample to be mixed with the known control sample in the same mass spectrometry run.

In one preferred embodiment, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In some embodiments the relative amounts of one or more biomolecules present in a first or second sample is determined, in part, by executing an algorithm with a programmable digital computer. The algorithm identifies at least one peak value in the first mass spectrum and the second mass spectrum. The algorithm then compares the signal strength of the peak value of the first mass spectrum to the signal strength of the peak value of the second mass spectrum of the mass spectrum. The relative signal strengths are an indication of the amount of the biomolecule that is present in the first and second samples. A standard containing a known amount of a biomolecule can be analyzed as the second sample to better quantify the amount of the biomolecule present in the first sample. In certain embodiments, the identity of the biomolecules in the first and second sample can also be determined.

RNA Detection Techniques

Any method for qualitatively or quantitatively detecting a biomarker, e.g., Pdx-1 RNA, e.g., mRNA, can be used. Detection of RNA transcripts can be achieved by Northern blotting, for example, wherein a preparation of RNA is run on a denaturing agarose gel, and transferred to a suitable support, such as activated cellulose, nitrocellulose or glass or nylon membranes. Radiolabeled cDNA or RNA is then hybridized to the preparation, washed and analyzed by autoradiography.

Detection of RNA transcripts can further be accomplished using amplification methods. For example, it is within the scope of the present disclosure to reverse transcribe mRNA into cDNA followed by polymerase chain reaction (RT-PCR); or, to use a single enzyme for both steps as described in U.S. Pat. No. 5,322,770, or reverse transcribe mRNA into eDNA followed by symmetric gap ligase chain reaction (RT-AGLCR) as described by R. L. Marshall, et al., PCR Methods and Applications 4: 80-84 (1994).

In one embodiment, quantitative real-time polymerase chain reaction (qRT-PCR) is used to evaluate mRNA levels of biomarker. A biomarker and a control mRNA levels can be quantitated in cancer tissue or cells and adjacent benign tissues.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; and target mediated amplification, as described by PCT Publication WO9322461.

Exemplary primers that can be used for amplification of Pdx-1 nucleic acid portions are set forth as: F: 5′-GGTGGAGCTGGCTGTCATGT-3′ (SEQ ID NO: 23); R: 5′-CGCGCTTCTTGTCCTCCTC-3′ (SEQ ID NO:3) and F: 5′-AAGTCTAACAAAGCTCACGCG-3′ (SEQ ID NO: 24); R: 5′-GTAGGCGCCGCCTGC-3′ (SEQ ID NO:4).

In situ hybridization visualization can also be employed, wherein a radioactively labeled antisense RNA probe is hybridized with a thin section of a biopsy sample, washed, cleaved with RNase and exposed to a sensitive emulsion for autoradiography. The samples can be stained with haematoxylin to demonstrate the histological composition of the sample, and dark field imaging with a suitable light filter shows the developed emulsion. Non-radioactive labels such as digoxigenin can also be used.

Another method for evaluation of biomarker expression is to detect gene amplification by fluorescent in situ hybridization (FISH). FISH is a technique that can directly identify a specific region of DNA or RNA in a cell and therefore enables to visual determination of the biomarker expression in tissue samples. The FISH method has the advantages of a more objective scoring system and the presence of a built-in internal control consisting of the biomarker gene signals present in all non-neoplastic cells in the same sample. Fluorescence in situ hybridization is a direct in situ technique that is relatively rapid and sensitive. FISH test also can be automated. Immunohistochemistry can be combined with a FISH method when the expression level of the biomarker is difficult to determine by immunohistochemistry alone.

Alternatively, mRNA expression can be detected on a DNA array, chip or a microarray. Oligonucleotides corresponding to the biomarker(s) are immobilized on a chip which is then hybridized with labeled nucleic acids of a test sample obtained from a subject. Positive hybridization signal is obtained with the sample containing biomarker transcripts. Methods of preparing DNA arrays and their use are well known in the art. (See, for example U.S. Pat. Nos. 6,618,6796; 6,379,897; 6,664,377; 6,451,536; 548,257; U.S. 20030157485 and Schena et al, 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the biological sample to be tested, reverse transcribed, and fluorescent-labeled cDNA probes are generated. The microarrays capable of hybridizing to a biomarker, e.g., Pdx-1, cDNA are then probed with the labeled cDNA probes, the slides scanned and fluorescence intensity measured. This intensity correlates with the hybridization intensity and expression levels.

Types of probes for detection of RNA include cDNA, riboprobes, synthetic oligonucleotides and genomic probes. The type of probe used will generally be dictated by the particular situation, such as riboprobes for in situ hybridization, and cDNA for Northern blotting, for example. Most preferably, the probe is directed to nucleotide regions unique to the particular biomarker RNA. The probes can be as short as is required to differentially recognize the particular biomarker mRNA transcripts, and can be as short as, for example, 15 bases; however, probes of at least 17 bases, more preferably 18 bases and still more preferably 20 bases are preferred. Preferably, the primers and probes hybridize specifically under stringent conditions to a DNA fragment having the nucleotide sequence corresponding to the target gene. As herein used, the term “stringent conditions” means hybridization will occur only if there is at least 95% and preferably at least 97% identity between the sequences.

The form of labeling of the probes can be any that is appropriate, such as the use of radioisotopes, for example, ³²P and ³⁵S. Labeling with radioisotopes can be achieved, whether the probe is synthesized chemically or biologically, by the use of suitably labeled bases.

The following examples are offered to more fully illustrate the disclosure, but are not to be construed as limiting the scope thereof.

### EXAMPLES

### Example 1

### Detection of Pancreatic Epithelial Cells Experimental Procedures

### Mouse Strains and Experimental Treatment

The behavior of mutant strains bearing various allele combinations of Pdx1-Cre KrasG12D, p16/p19fl, and p53fl has been described previously (Aguirre et al., 2003; Bardeesy et al., 2006; Hingorani et al., 2003; and U.S. patent application Ser. No. 11/503,499, the contents of which are expressly incorporated herein by reference). To perform lineage tracing, a RosaYFP reporter allele was introduced into these mutant backgrounds to generate a panel of compound mutant strains: Pdx1-Cre; RosaYFP (“CY”), Pdx 1-Cre; KrasG12D; RosaYFP (“KCY”), Pdx1-Cre; KrasG12D; p16/p19fl/+; RosaYFP (“IKCY”), and Pdx1-Cre; KrasG12D; p53fl/+; RosaYFP (“PKCY”). All experiments involving the KCY model employed mice between 8 and 10 weeks of age. For studies involving mice harboring only PaniN lesions by histologic analysis (“PanIN” mice), PKCY animals were sacrificed at 8-10 weeks of age based on prior observations regarding tumor progression (Bardeesy et al., 2006); no PKCY mice (out of 18 examined) had evidence of carcinoma at this time point. For PDAC mice, animals were examined three times per week for evidence of morbidity and sacrificed when they exhibited limited physical activity, depressed response to toe pinch, dehydration, and/or abdominal enlargement from ascites. More than 90% of IKCY and PKCY mice were 34-38 weeks of age or 16-20 weeks of age, respectively, at the time of sacrifice.

Experimental pancreatitis was elicited with cerulein as described (Siveke et al., 2008), and specimens were obtained after 3 days of treatment. Three to four CY, KCY, PanIN PKCY, and PDAC PKCY were used for cerulein and vehicle (PBS) treated cohorts. Orthotopic transplantations were performed on NOD/SCID mice by injecting 1×10⁵ sorted pancreas cells as previously described (Mohammad et al., 1998). PKCY PanIN mice aged 10 weeks were treated with seven daily injections of dexamethasone or vehicle (DMSO) as described (Stairs et al., 2011) and analyzed 24 hours later.

### Cell Staining

For experiments involving flow cytometry and sorting of pancreatic cells, at least 25% of the tissue was saved for histologic analysis and was processed and stained. To create a single cell suspension, pancreatic tissue was rinsed vigorously in cold DMEM/F12 three times before mincing with scissors (approximately 100 chops). The minced pieces were then incubated in preheated collagenase with protease inhibitors (2 mg/ml; Sigma) for 20 min at 37° C. Vigorous vortexing was performed every 5 min during this incubation. The dissolved pieces were then poured over a 40 mM cell strainer. The flow through was resuspended in cold 2% FCS/DMEM/F12, centrifuged, washed once, and kept on ice in the dark. Enriched blood samples or dissociated pancreatic specimens were stained for various markers prior to flow cytometric analysis (FACS Calibur, BD, or Accuri C6, Accuri Cytometers) or flow sorting (FACSVantage with FACSDiva option, BD). For analysis and sorting based on endogenous YFP and surface markers, primary antibodies (1:100) and secondary antibodies (1:50) were incubated with cells in 10% FCS/DMEM/F12 for 20 min at 4° C. Primary PanIN cell lines from IKCY and PKCY mice were used as positive controls. Isotype controls were also run. For intracellular staining of CPCs and pancreas cells, cells were washed with 10% FCS/DMEM/F12 before washing in freshly prepared 13fixation/permeabilization solution (Fix/Perm; eBioscience) followed by incubation in Fix/Perm for 30 min in the dark at 4° C. Cells were washed twice with 13 permeabilization buffer (Perm; eBioscience) before incubation in Block-P solution (10% FCS in freshly prepared Perm). After 15 min at 4° C., primary antibodies were added and incubated for an additional 20 min on ice. Secondary antibodies in Block-P were incubated with the cells for 20 min on ice. Cells were washed twice in Block-P before resuspension in 5% FCS/PBS.

### Pancreatosphere Assay

YFP⁺ blood or pancreatic cells from the same mouse were sorted into ultra-low attachment 96-well plates (Corning) at 1 cell per well, confirmed by microscopy. Cells were grown as previously described (Rovira et al., 2010) and assayed at 5 days for clonal growth of fluorescent cells (defined as clusters of cells >3 cell widths in diameter) or presence of live YFP⁺ cells (singlets or doublets). Three to four mice were analyzed for each category.

### Additional Mouse Strains

The RosaYFP reporter allele was bred into KrasG12D; Mist1CreERT2 mice (Habbe et al., 2008). Tamoxifen induction of Cre recombinase expression was performed in Mist1CreERT2; K-rasG12D; RosaYFP mice at 6 weeks of age by three daily intraperitoneal injections of tamoxifen (5 mg/day, Sigma). Three noninduced littermates were used as control animals at each time point. Chronic pancreatitis was induced in these mice with cerulein (10 mg/injection, three daily injections three times per week for 3 weeks).

### Phlebotomy

To extract blood cells, a midline laparotomy was performed to expose the abdomen, and a 27 g needle attached to a 1 ml syringe prefilled with 0.1 ml chilled Lithium heparin (1 mg/ml) was inserted into either the right atrium/right ventricle or left atrium/left ventricle immediately postmortem. Steady negative pressure was applied until a second syringe was needed. Extracted blood volume was estimated using gradations on the syringe before placement into heparin-lined tubes on ice. Blood samples were subjected to negative selection of leukocytes and erythrocytes using the EasySep Mouse Epithelial Cell Enrichment Kit (Stem Cell Technologies) or FACS Lyse buffer (BD), as recommended by the manufacturer. After enrichment, remaining cells were resuspended in 10% FCS/DMEM/F12 for further analysis.

### Mouse Surgery

Orthotopic transplantations were performed on NOD/SCID mice (Jackson Laboratories). After induction of anesthesia using isoflurane, the mouse was transferred to the surgical field and a nose cone fitted for constant isoflurane anesthesia. After depilation and sterile preparation of the abdomen using rubbing alcohol and iodine, a small (10 mm) incision was made over the left upper quadrant of the abdomen. After exposure of the peritoneal cavity, the tail of the pancreas, which was adherent to the spleen, was then located. Pancreas cells (1 3 105) suspended in 50 ml of cold DMEM/F12 were then injected into the tail of the pancreas via a 27 g needle over the span of 10 s. Visualization of a bleb with minimal fluid leakage confirmed successful injection. The ventral incision was then closed with 4-0 silk suture in a double layer using a running suture technique. Following surgery mice were given buprenorphine at a dose of 0.05-0.1 mg/kg every 12 hr for 48 hr. Main pancreatic duct ligations were performed on 8-10 week PKCY mice as previously described (Scoggins et al., 2000). Mice were then sacrificed for analysis 1 week after surgery. Control mice were subjected to a “Sham” surgery where a laparotomy was performed, and the pancreas was exposed, but no ligations were made.

### RNA and DNA Extraction and OCR

Cells destined for transcriptional analysis were sorted into either RLT buffer (QIAGEN) or Trizol LS Reagent (Invitrogen). RNA was extracted from samples >1,000 cells using the RNEasy kit (QIAGEN). RNA from samples with fewer cells, including all sorted blood cell samples, was isolated using the PicoPure Extraction kit as directed (Arcturus). qPCR was performed as previously described (Zong et al., 2009), and primary sequences used are listed in Table 1, below. Relative expression was determined after adjusting for GAPDH. Genomic DNA was extracted from sorted CPCs pooled from three tumor-bearing mice using phenol or DNeasy kit (QIAGEN).

TABLE 1
Sequences of primers used for quantitative PCR.

| | Forward | Reverse |
|---|---|---|
| E-cad | AGGTTTTCGGGCACCACTTA | TGATGTTGCTGTCCCCAAGT |
| | (SEQ ID NO: 5) | (SEQ ID NO: 6) |
| CK19 | TCCCAGCTCAGCATGAAAGCT | AAAACCGCTGATCACGCTCTG |
| | (SEQ ID NO: 7) | (SEQ ID NO: 8) |
| Zeb1 | CCACTGTGGAGGACCAGAAT | CTCGTGAGGCCTCTTACCTG |
| | (SEQ ID NO: 9) | (SEQ ID NO: 10) |
| Fsp-1 | TTGTGTCCACCTTCCACA | GCTGTCCAAGTTGCTCAT |
| | (SEQ ID NO: 11) | (SEQ ID NO: 12) |
| N-cad | CATCAACCGGCTTAATGGTG | ACTTTCACACGCAGGATGGA |
| | (SEQ ID NO: 13) | (SEQ ID NO: 14) |
| YFP | GAAGTTCATCTGCACCACCG | GGTA GCTCAGGTAGTGGTT |
| | GCAAG | GTCG |
| | (SEQ ID NO: 15) | (SEQ ID NO: 16) |
| K-ras | CGCGCCATTTCGGACCCGGA | CGTCAAGGCGCTCTTGCCTA |
| | | CG |
| | (SEQ ID NO: 17) | (SEQ ID NO: 18) |
| GAPDH | ATGTTCCAGTATGACTCCAC | GAAGACACCAGTAGACTCCA |
| | TCACG | CGACA |
| | (SEQ ID NO: 19) | (SEQ ID NO: 20) |

### Pancreas Histology

Independent analysis of PanIN pancreases for evidence of cancer was performed at Johns Hopkins University School of Medicine using H&E analysis of at least three widely spaced sections. PanIN lesions were independently graded and confirmed in 34 random samples by a pancreatic pathologist. For histologic examination, pancreata, tumors, livers, and lungs were removed and washed three times in cold PBS before fixation in Zinc Formalin (Polysciences Corp) for 2 hr, dehydration, and paraffin embedding.

### Immunohistochemistry and Immunofluorescence

Immunostaining was performed as previously described (Zong et al., 2009). Cell counting was performed on digital images and results were averaged. Experiments involving the survey of liver sections for evidence of colonization employed 10 mM thick sections. Five adjacent sections were cut and then 50 mm was skipped before the next five sections were cut. This was repeated six times. Sections from each group were analyzed for YFP+ cells. For multicolor immunofluorescence staining, Zn-formalin fixed, paraffin-embedded sections were deparaffinized and rehydrated before antigen retrieval in R-buffer A in a slide steamer (Electron Microscopy Sciences). After approximately 2 hr, slides were washed in PBS and blocked for 1 hr at room temperature (RT) in blocking buffer (5% FCS/0.3% Triton X-100/PBS). Primary and secondary antibodies were suspended in blocking buffer and incubated on slides for 1 hr at RT with two washes in PBST for 5 min in between and after the secondary antibody incubation. DAPI was included in the secondary antibody solution. Coverslips were mounted using Aqua-Mount (Electron Microscopy Sciences) and visualized using an Olympus IX71 inverted multicolor fluorescent microscope. IF from pancreases from in KrasG12D; Mist1CreERT2; RosaYFP mice were visualized using a Nikon Eclipse T7 Confocal Microscope and analyzed using NIS Elements Software AR 3.2.

For Pdx1 immunohistochemistry, paraffin sections of human pancreata were obtained from the Human Tissue Bank of the Morphology Core within the Penn Center for Molecular Studies in Digestive and Liver Diseases. Sections were deparaffinized, rehydrated, and quenched in H2O2 before blocking in 5% donkey serum in PBS for 1 hr at RT. Slides were then incubated with Pdx1 antibody for 1 hr at RT, washed three times, and incubated with biotinylated donkey anti-goat secondary antibody. Slides were then stained using the ABC reagent according to manufacturer's instructions (Vector Laboratories) and immediately washed after 1-2 min. Slides were counterstained with hematoxylin for 20 s. Tissues from a human pancreatic tissue microarray (Johns Hopkins University Department of Pathology) were similarly stained with Pdx1 antibody. Antibodies used are listed in Table 2.

TABLE 2

| | Concentration | | |
|---|---|---|---|
| Antibody | IF | FACS | Company |
| Goat and Chicken α | 1:500 | 1:100 | Abcam |
| GFP | | | |
| Rabbit α Zeb1 | 1:200 | 1:100 | Santa Cruz |
| Rat α E-cadherin | 1:1000 | 1:250 | Invitrogen/Zymed and |
| | | | R&D Systems |
| | | | (fluorophore conjugated) |
| Rabbit α FSP-1 | 1:500 | 1:100 | Dako |
| (S100A4) | | | |
| Rabbit α CK19 | 1:1000 | 1:250 | Gift from Douglas Melton |
| Rat α EpCAM | 1:200 | 1:50 | DSHB and R&D Systems |
| | | | (fluorophore conjugated) |
| Mouse α N-cadherin | 1:250 | n/a | BD Biosciences |
| Goat and Rat α CD45 | n/a | 1:50 | R&D systems, BD |
| | | | Biosciences (fluorophore |
| | | | conjugated), Stem Cell Tech |
| Rat α Ter-119 | n/a | 1:50 | BD Biosciences, Stem Cell |
| | | | Tech |
| Rat α CD24 | n/a | 1:50 | BD Biosciences |
| | | | (fluorophore conjugated) |
| Rat α CD44 | n/a | 1:50 | BD Biosciences |
| | | | (fluorophore conjugated) |
| Goat α Pdx-1 | 1:1000 | 1:100 | Abcam and R&D Systems |
| | | | (fluorophore conjugated) |
| Rabbit α Snail1 | 1:100 | n/a | Abcam |
| Rabbit α Slug | 1:500 | n/a | Gift from Joel Habener |
| Goat α Sip1 | 1:500 | n/a | Santa Cruz |
| Rabbit α GFP | 1:100 | n/a | Invitrogen |
| Mouse α E-cadherin | 1:50 | n/a | BD Pharmingen |

### In Vitro Studies

Primary cell lines were derived from PanIN mice after sorting pancreas single-cell suspensions for YFP⁺ cells. Sorted YFP⁺ cells were then placed in six-well plates in pancreatic ductal cell media (Schreiber et al., 2004) and passaged at 80% confluence, Cells were transferred to multiple 6-well plates (250,000 cells per well) in pancreatic ductal cell media containing DMSO vehicle, cerulein (100 nM, Sigma), dexamethasone (Dex, 1 mM, Sigma), TGF-b (5 ng/ml, Peprotech), or Dex (1 mM) and TGF-b (5 ng/ml). Morphology was photodocumented at 6 hr, 24 hr, and 48 hr after treatment using an inverted microscope. After 48 hr of exposure, cells were counted and RNA isolated, cDNA synthesized, and qPCR performed as described above.

### Kras Sequencing

To determine whether CPC contained the recombined oncogenic Kras allele, YFP⁺ cells were sorted from the blood of 8- to 10-week old PKCY mice and RNA isolated and eDNA synthesized as above. Kras cDNA was amplified using high cycle PCR (45 cycles, annealing temperature at 60° C.) using primers which flanked codon 12 (forward: CGCGCCATTTCGGACCCGGA (SEQ ID NO:21); reverse: CGTCAAGGCGCTCTTGCCTACG) (SEQ ID NO:22), the location of a single G/D point mutation in IKCY and PKCY mice (Tuveson et al., 2004). The products of this reaction (161 bp) were clarified on a 2% agarose gel, removed, extracted using a gel extraction kit (QIAGEN), and sequenced.

### Statistical Analysis

All tests for significance, unless otherwise denoted, featured unpaired Student's t test. All statistical analyses, including two-way ANOVA, were performed using GraphPad Prism 5 (GraphPad Software).

### Results

### Enhanced Detection of EMT Using Epithelial Lineage Tracing

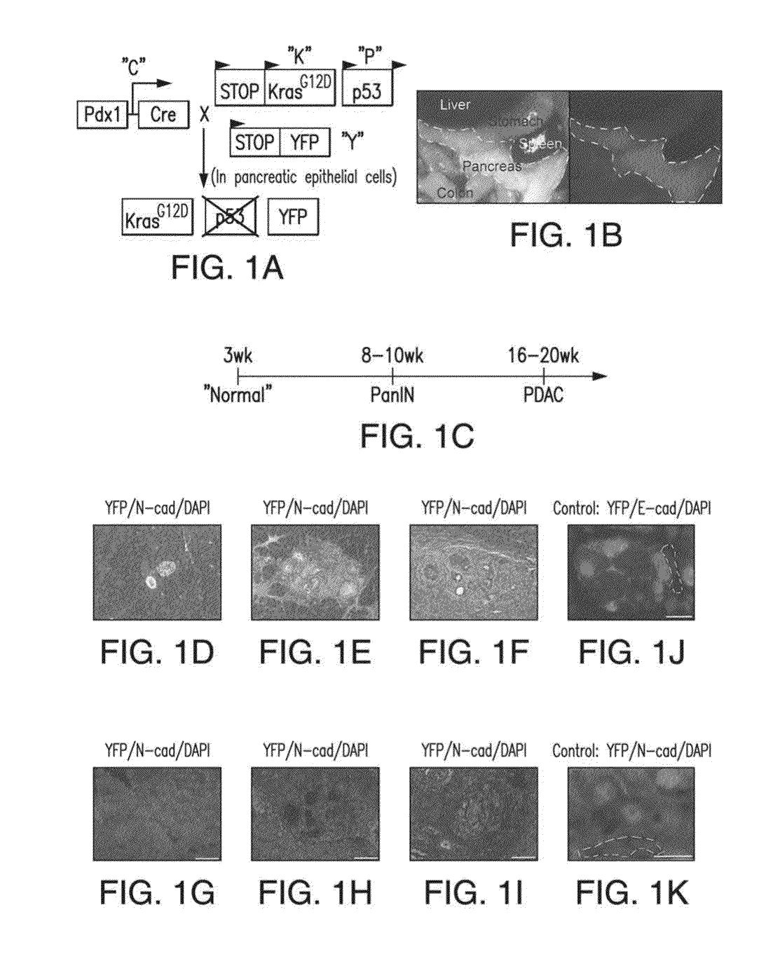

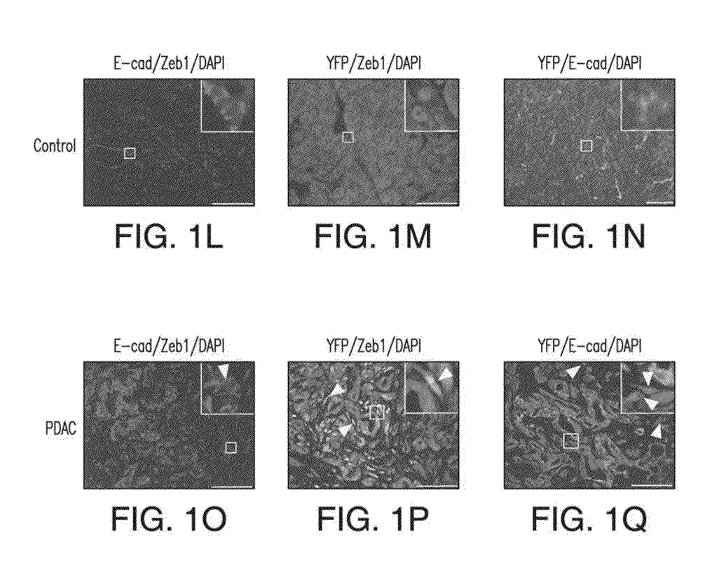

A Cre-lox-based mouse model of PDAC was used to study the fate of pancreatic epithelial cells during various stages of tumor progression (Bardeesy et al., 2006). The model relies on the Pdx1-Cre transgenic strain (Gu et al., 2003) to generate pancreas-specific mutations in Kras and p53, genes that are mutated with high frequency in human pancreatic cancers (Hezel et al., 2006). In order to track pancreatic epithelial cells during tumor progression, a RosaYFP allele was introduced into the mutant background, resulting in highly specific and efficient (>95%) labeling (FIGS. 1A and 1B). Animals containing all four alleles were referred to as PKCY mice. A second model, in which a single allele of p16^{Ink4a/Arf} was deleted in place of p53 (IKCY; Aguirre et al., 2003), was also employed and yielded similar results. The lineage-labeled mouse models displayed similar histology as nonlabeled models, including the development of pancreatic intraepithelial neoplasias (PanINs), primary tumors, and metastases, with reproducible kinetics (FIGS. 1C-1I). Because the Pdx1 promoter is active only in endoderm-derived pancreatic cells (Gu et al., 2003), only the epithelium was tagged by this method. Importantly, mesenchymal cells were never labeled under control conditions in Pdx1⁻Cre; RosaYFP (CY) animals (FIGS. 1J and 1K).

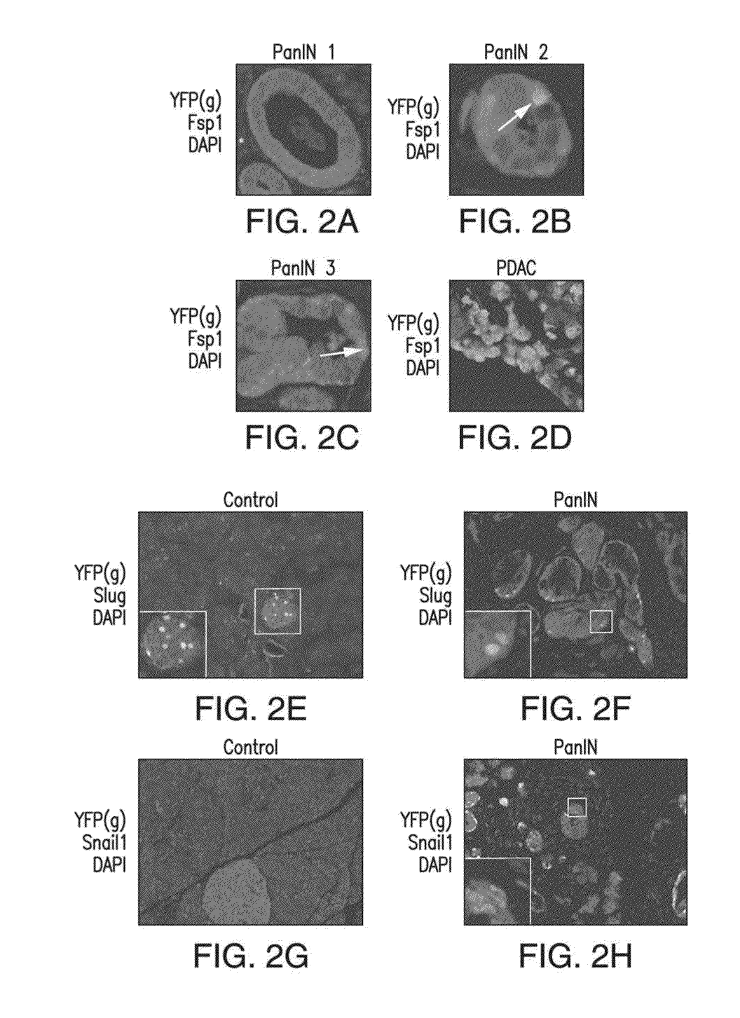

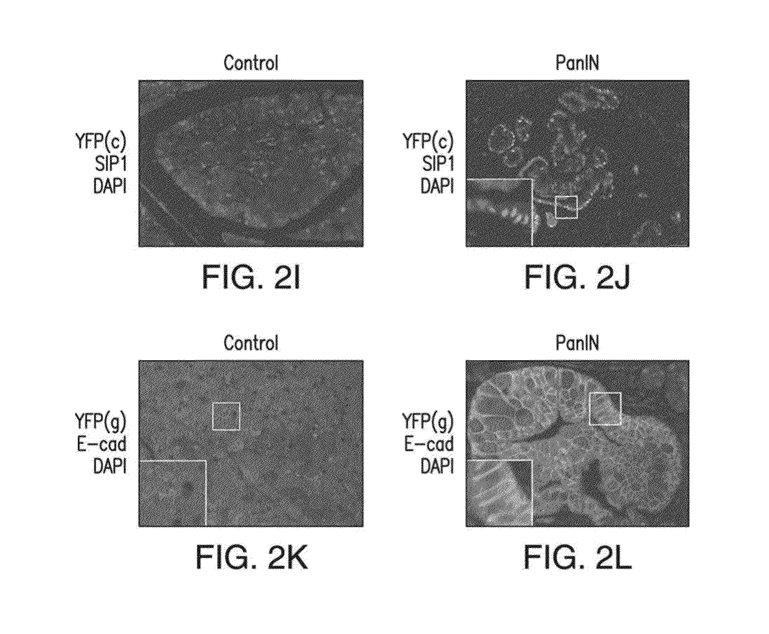

Initially, cancer cells that coexpressed an epithelial marker and a mesenchymal marker were targeted, a standard approach used to detect cells at an “intermediate stage” of EMT. In the course of these studies, several mesenchymal markers were used (FIG. 2) but analysis was focused on Zeb1 and Fsp1, as these markers serve as independent predictors of mortality in subjects with pancreatic cancer (Brabletz et al., 2011; Wang et al., 2007). Using this method, tumor cells were detected in tumor-bearing mice (“PDAC mice”) that coexpressed either Zeb1 or Fsp1 and the epithelial marker E-cadherin (E-cad; FIG. 1O), indicating that such “biphenotypic” cells exist, albeit at a low frequency (<10%).

The YFP lineage label was then used to identify PDAC cells that had completed an EMT. Because labeling was limited to cells of epithelial origin, EMT was defined as having occurred if a cell coexpressed YFP and either Zeb1 (FIG. 1P) or Fsp1 (FIG. 2D) and/or lacked E-cad (FIG. 1Q) expression. Using this approach, it was observed that 42% of the lineage-labeled YFP⁺ cells in PKCY tumors had undergone EMT (FIG. 1P); higher rates of EMT (68% of all YFP cells) were found in the IKCY model. EMT was not detected in lineage-labeled CY control mice by either method (FIG. 1L-1N). Thus, genetic lineage marking is a sensitive tool for distinguishing cells of epithelial and mesenchymal origin and for the detection of EMT.

### EMT in Premalignant Lesions

EMT has been proposed to be a prerequisite for invasion and dissemination of carcinoma cells Manahan and Weinberg, 2011). To determine when EMT first occurs during PanIN-to carcinoma progression, 8- to 10-week-old PKCY mice were analyzed. At these time points, only precancerous PanIN lesions were present, and there was no histological evidence of PDAC based on extensive H&E analysis (n=18); these animals were referred to as “PanIN mice” solely to reflect the histological state of the pancreas at these time points.

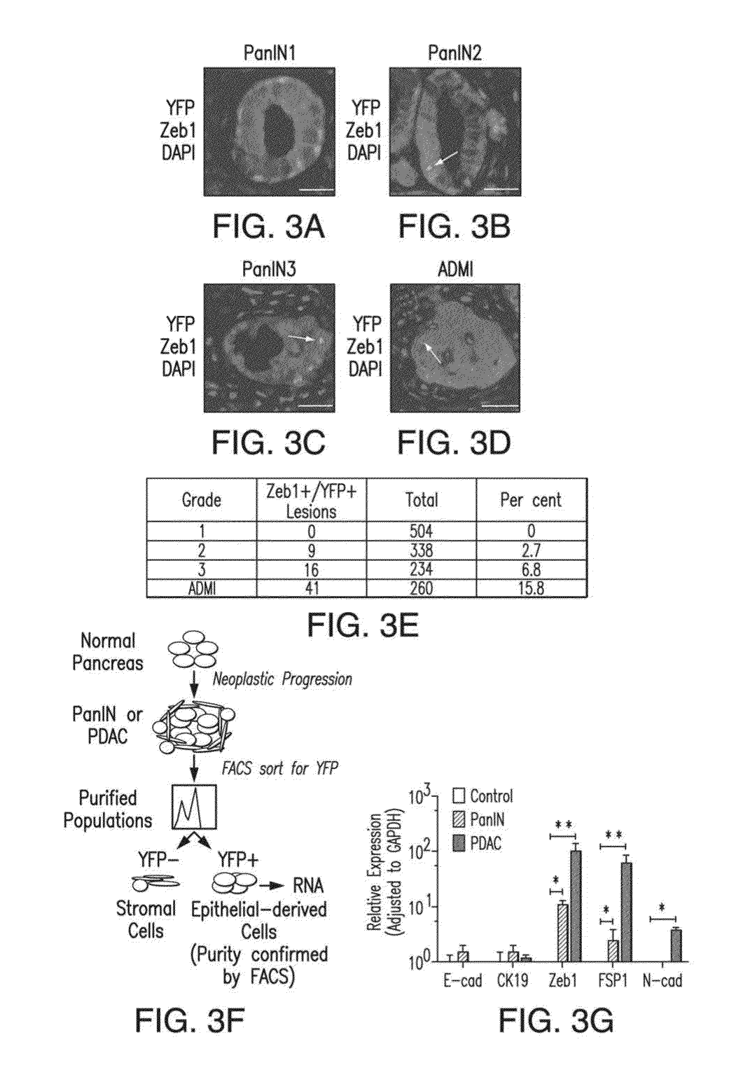

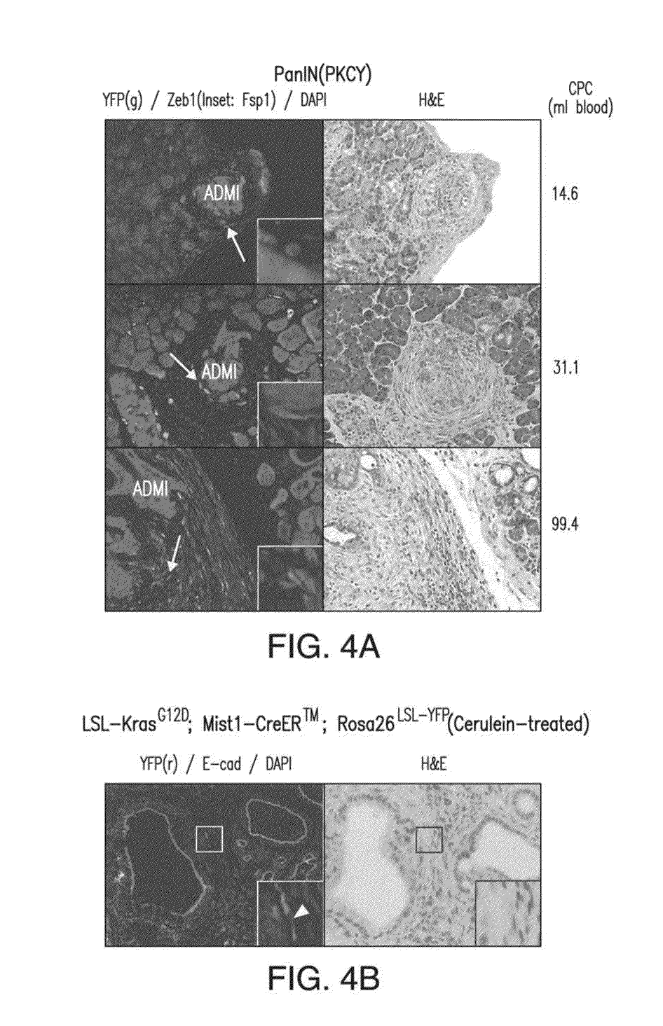

EMT was identified in premalignant lesions from both models (FIGS. 3B, 3C, 2B, and 2C). 2.7% and 6.8% of all PanIN 2 and 3 lesions, respectively, contained at least one YFP⁺Zeb1⁺ cell, whereas EMT was never observed in PanIN 1 lesions (FIGS. 3A and 3E). Similar results were noted with other mesenchymal markers, including Fsp1, Slug, Snail1, and Sip1 (FIG. 2). EMT was also prevalent in areas of acinar-to-ductal metaplasia (ADM), particularly in lesions surrounded by abundant inflammatory cells (FIGS. 3D and 4A). These areas are referred to as ADMIs (acinar-to-ductal metaplasia with inflammation) and it was determined that 15.8% of ADMIs had evidence of EMT in 8- to 10-week-old PKCY PanIN mice (FIG. 3E).

YFP⁺ cells were sorted and qPCR was performed to confirm that epithelium-derived pancreatic cells activated a mesenchymal program at the transcriptional level (FIG. 3F). Transcripts for Zeb1, Fsp1, and N-cadherin were found in YFP⁺ cells from tumor-bearing PKCY animals and PanIN animals but not in YFP⁺ cells from CY control mice (FIG. 3G; p<0.01). These data indicate that EMT occurs in PanIN lesions and ADMIs prior to tumor formation.

### Pancreatic Epithelial Cells Spread Before Tumor Formation

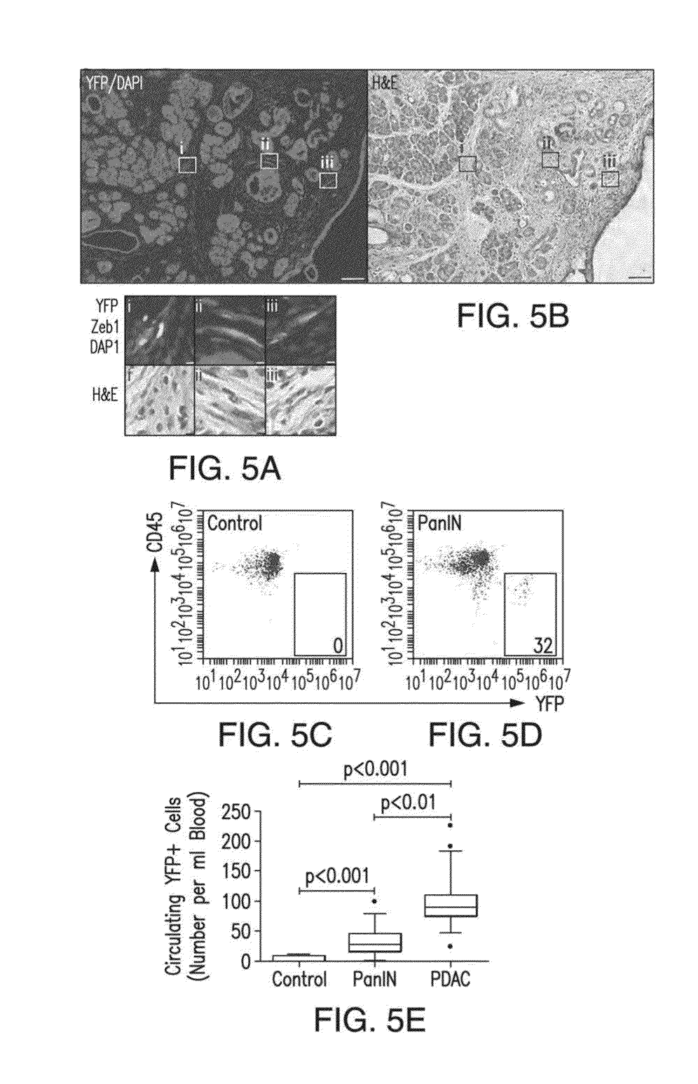

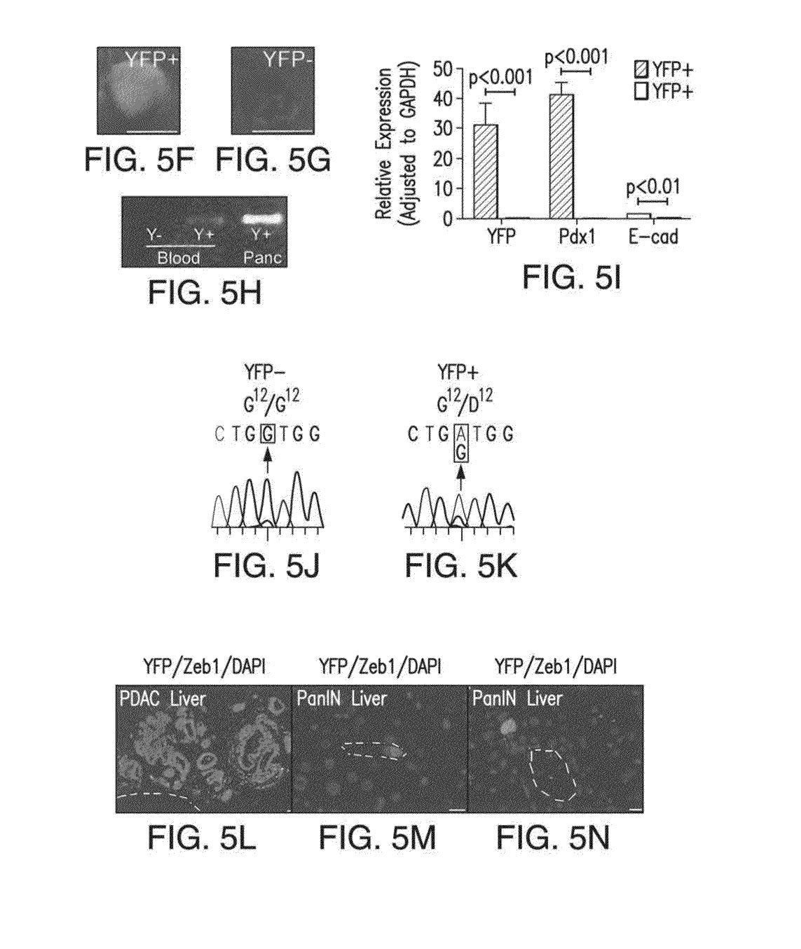

Cells that have undergone EMT acquire an invasive phenotype in vitro (Polyak and Weinberg, 2009). Thus, it was hypothesized that cells that undergo EMT in PanIN mice might also have invasive properties. Consistent with this notion, individual YFP⁺ cells were identified that had traversed the basement membrane and dissociated from any discernible pancreatic epithelial structure (a process referred to herein as “delamination”) in mice bearing PanIN 2 and PanIN 3 lesions (FIGS. 5A and 4A). Most of these cells expressed Zeb1 (FIG. 5A, i-iii) and had acquired a fibroblast-like morphology, making them indistinguishable from surrounding stromal cells by conventional histology (FIGS. 5A, 5B, and 4); a fraction of the delaminated YFP⁺ cells also expressed Fsp1 (FIG. 4A, insets). To rule out the possibility that the Pdx1 promoter might be ectopically activated in mesenchymal cells during premalignant progression, lineage tracing was performed in Mist1^{CreERT2} mice whose acinar cells had been labeled by tamoxifen pulse. In the setting of experimental pancreatitis (as described in greater detail below), mist1^{CreERT2}; Kras^{G12D}; Rosa^{YFP} pancreata contained fibroblast-like YFP⁺ cells that lacked E-cad expression (FIG. 4B). Because Mist1^{CreERT2} mediates labeling solely at the time of tamoxifen administration (Habbeet al., 2008), this experiment demonstrates unambiguously that the labeled mesenchymal cells were derived from pancreatic acinar cells in vivo.

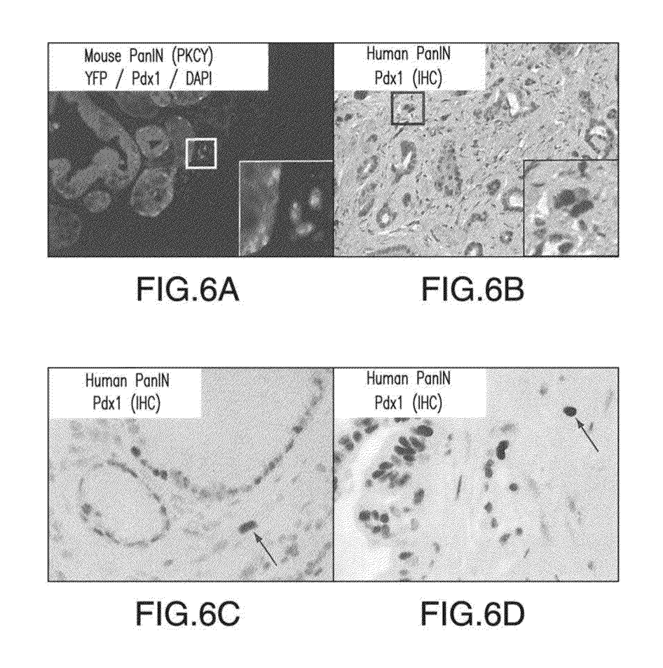

To extend these studies, immunostaining for the Pdx1 transcription factor was performed. Pdx1 is normally expressed at high levels during pancreatic development and in adult b cells, and it is commonly “reactivated” in human PanIN lesions and in PDAC (Park et al., 2011). Pdx1 was widely expressed in PanIN lesions and in a subset of YFP⁺ cells that had delaminated in PanIN mice (FIG. 6A). Consistent with these data, sections of human pancreas that contained PanIN lesions (but no tumor) exhibited scattered Pdx1⁺ cells that were separated from any defined epithelial structure (FIGS. 6B-6D). Thus, human pancreatic cells can delaminate from PanIN lesions as they do in the mouse model.

Because lineage tracing demonstrated that pancreatic cells can cross the basement membrane before invasive behavior is detectable by standard histology, it was asked whether these cells could also enter the bloodstream prior to tumor formation. In tumor-bearing PDAC mice, YFP⁺ circulating pancreatic cells (CPCs) were readily detected in the blood by flow cytometry (FIG. 5E). Surprisingly, CPCs were also abundant in the bloodstream of 8- to 10-week-old PKCY PanIN mice (FIGS. 5C-5G). Sorted YFP⁺ cells contained the recombined YFP allele (FIG. 5H), expressed transcripts for YFP, Pdx1, and E-cad (FIG. 3I), and carried the Gly/Asp mutation at codon 12 of the Kras cDNA (FIGS. 5J and 5K). Thus, cells derived from the pancreatic epithelium are present in the circulation of mice with no evidence of carcinoma.

These data raised the possibility that CPCs from PanIN mice might seed distant organs. To assess this possibility, PDAC mice were first examined as a positive control. Bright-field stereomicroscopy permitted detection of liver and lung metastases in 8/20 animals; use of the YFP lineage label enhanced detection, revealing micrometastases in 16/20 PDAC mice (FIG. 5L). Next, 8- to 10-week-old PKCY PanIN mice were analyzed. Although no animals had macro- or micro-metastases, liver seeding by YFP⁺ cells was detected in 4/11 PanIN mice (FIGS. 5M and 5N); most were single cells located near blood vessels and expressed neither Zeb1 nor E-cad (FIGS. 5M and 5N). By contrast, 0/10 livers from lineage-labeled control CY mice harbored YFP^{S} cells when examined by the same technique.

### Characterization of CPCs

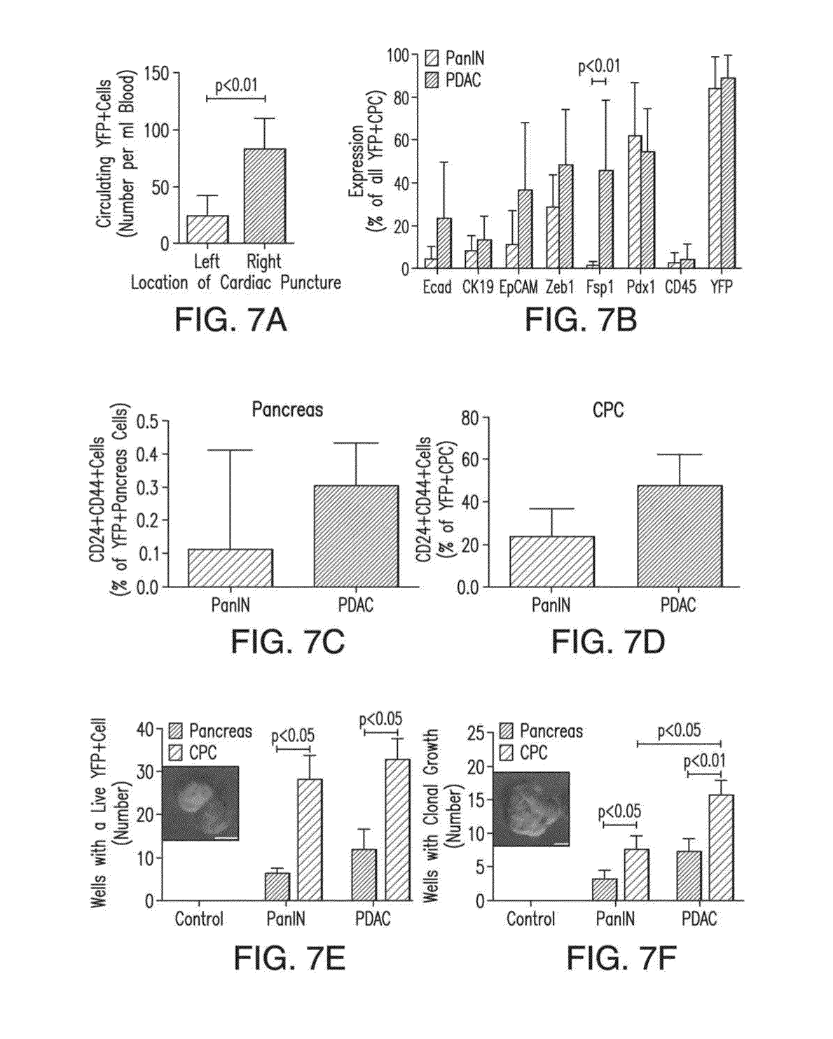

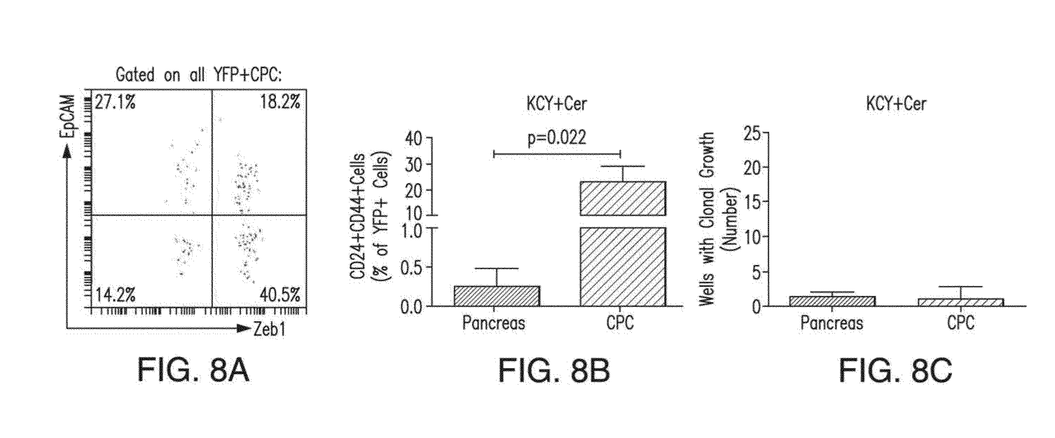

The number of YFP⁺ CPCs from PDAC mice depended on the location of blood collection, with a roughly 3-fold increase in abundance in the right side of the heart compared to the left side (FIG. 7A). Only 2/9 PDAC mice had evidence of lung metastases, suggesting that the vast majority of CPCs do not survive passage through the pulmonary circulation. To determine whether cells in the circulation exhibited an epithelial, mesenchymal, or mixed phenotype, CPCs were stained with a variety of markers in a flow cytometric assay. The epithelial markers E-cad, CK19, and EpCAM were detected in fewer than 20% of PanIN CPCs and fewer than 40% of PDAC CPCs (FIG. 7B). With the exception of Fsp1—which was detected in only 1.1% of all PanIN CPCs compared to 45.2% in PDAC CPCs (p<0.01)—there were no statistically significant differences in cell-surface phenotype between CPCs from the two groups (FIG. 7B). Coimmunofluorescence for Zeb1 and EpCAM revealed that approximately 40% of PDAC CPCs were Zeb1⁺, 27% were EpCAM⁺, and 18% were double positive (FIG. 8), suggesting that most CPCs do not exhibit a “mixed” epithelial-mesenchymal phenotype. These data indicate that CPCs from PDAC and PanIN animals are phenotypically similar and that a large fraction maintains a mesenchymal phenotype in the circulation.

### CPCs Exhibit Features of Cancer Stem Cells

Cancer stem cells are functionally defined as cells that have enhanced tumor-initiating capacity upon transplantation into a permissive host. In human pancreatic tumors, this activity can be contained within a CD24⁺CD44⁺ population of cells, among others (Hermann et al., 2007; Jimeno et al., 2009; Li et al., 2007). Because EMT in primary cells has been associated with the acquisition of stem cell-like characteristics (Mani et al., 2008), it was hypothesized that CPCs might also exhibit features of cancer stem cells. The relative abundance of CD24⁺CD44⁺ cells in pancreata and CPCs from PanIN and PDAC mice were compared. By fluorescence-activated cell sorting (FACS) analysis, 0.11%±0.32% and 0.30%±0.13% of sorted YFP⁺ cells from PanIN and PDAC pancreata, respectively, expressed both CD24 and CD44 (FIG. 7C). By contrast, 23.1%±12.9% and 46.4%±14.7% of sorted YFP⁺ CPCs from PanIN and PDAC samples were found to be CD24⁺CD44⁺, representing a greater than 100-fold enrichment when compared to the source pancreas (FIG. 7D).

The survival and self-renewal properties of CPCs directly was next assessed by employing an in vitro pancreatosphere assay, in which single YFP⁺ cells were cultured in attachment-free conditions (Rovira et al., 2010). In both PanIN and PDAC mice, YFP⁺ CPCs had significantly greater rates of clonal survival and growth compared to YFP+ pancreas cells from the same animal (FIGS. 7E and 7F; p<0.05). Taken together, these data indicate that during tumor evolution in vivo, bloodstream entry is associated with enrichment of cells that have phenotypic and functional features associated with pancreatic cancer stem cells.

Cells that have Undergone EMT have Tumor-Initiating Properties

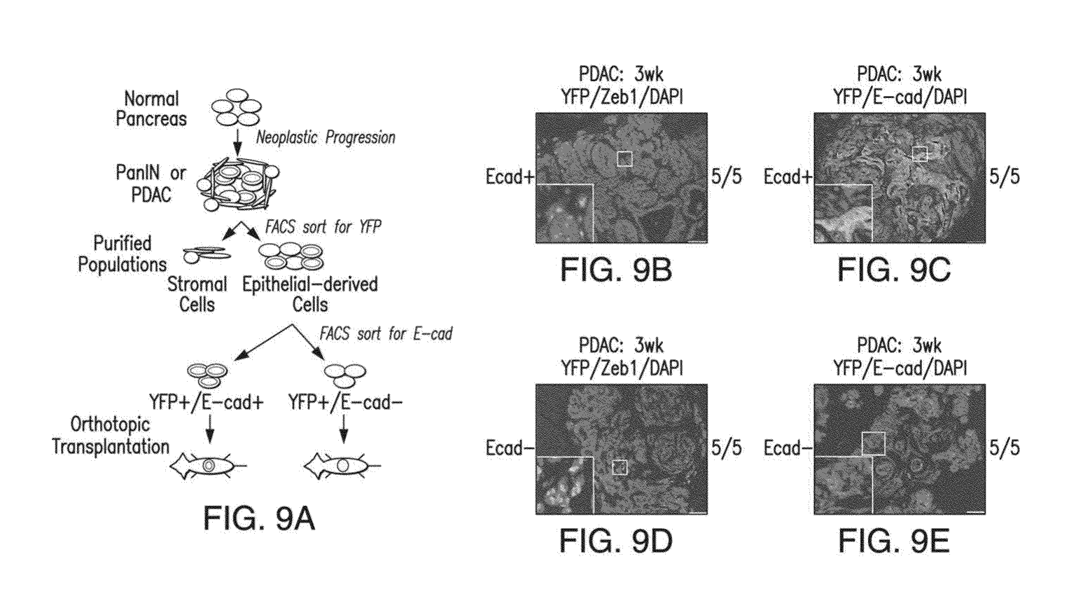

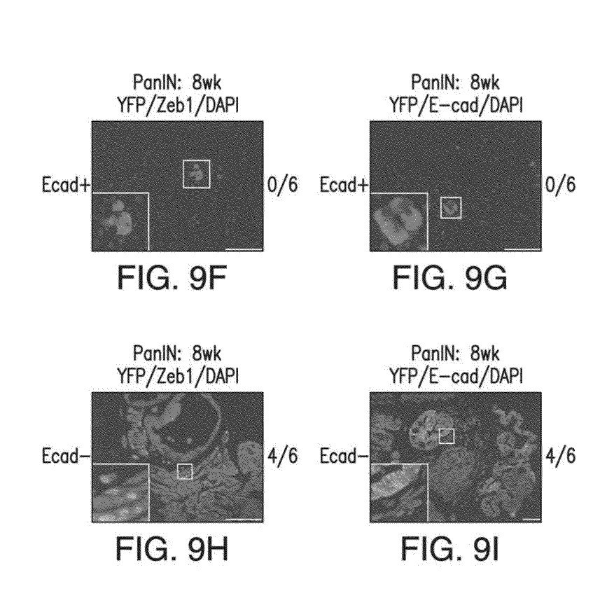

Although previous work has suggested a link between EMT and increased tumor aggressiveness, most studies have relied on in vitro manipulation of cancer cell lines to induce EMT (Weinberg, 2008). Such treatments could have a direct effect on cell behavior apart from their EMT-inducing activity, and thus a direct demonstration of the role of EMT in tumor progression is lacking. The described lineage-labeling system was used to isolate cells that had either lost or retained E-cadherin expression to determine whether an EMT in vivo is associated with tumor-initiating capacity (FIG. 9A). First, we transplanted 100,000 YFP⁺E-cad⁺ or YFP⁺E-cad⁻ pancreatic cells from PDAC mice into the pancreata of NOD/SCID animals (n=5 for each group). After 3 weeks, all transplants gave rise to large tumors with local invasion and distant metastasis regardless of E-cad status at the time of transplantation; tumors were histologically similar, and YFP⁺ cells coexpressing either Zeb1 or E-cad were found at comparable proportions in both groups (FIGS. 9B-9E). This result suggests that tumor derived E-cad⁺ and E-cad⁻ cells can each form tumors and that there is significant plasticity between epithelial and mesenchymal states.

By contrast, a dramatic effect of E-cad status on tumor formation was observed when cells from PanIN mice were transplanted: 4/6 animals transplanted with 100,000 YFR⁺E-cad⁻ cells formed tumors after 2 months, whereas 0/6 animals transplanted with 100,000 YFP⁺E-cad⁺ cells formed tumors over the same time period (FIGS. 9F-9I). Tumors derived from E-cad_ PanIN cells were heterogeneous with respect to E-cad and Zeb1 expression (FIGS. 9H and 9I). By comparison, E-cad⁺ transplanted animals had few detectable YFP⁺ cells at the 2 month time point, and almost all of the YFP⁺ cells detected were Zeb1⁻ (FIG. 9F). Mice transplanted with E-cad⁺ PanIN cells eventually developed tumors (with a mean latency of 4 months after transplantation) that were indistinguishable from the PDAC-derived tumors. Thus, functional analysis of tumor-initiating capacity revealed that E-cad⁻ cells that have undergone EMT have a marked advantage at the PanIN stage.

### Inflammation Promotes EMT, Invasiveness, and Dissemination

The emergence of PanIN lesions is associated with the appearance of an inflammatory stroma characterized by activated fibroblasts and myeloid-derived cells (Aguirre et al., 2003; Clark et al., 2007). Inflammation is commonly correlated with tumor initiation and progression (Coussens and Werb, 2002; Grivennikov et al., 2010) and accelerates pancreatic carcinogenesis in adult Kras mutant mice (Guerra et al., 2007, 2011). The observation that ADMIs have a high prevalence of EMT (FIG. 5) led to the hypothesis that inflammation contributes to EMT and dissemination at the PanIN stage.

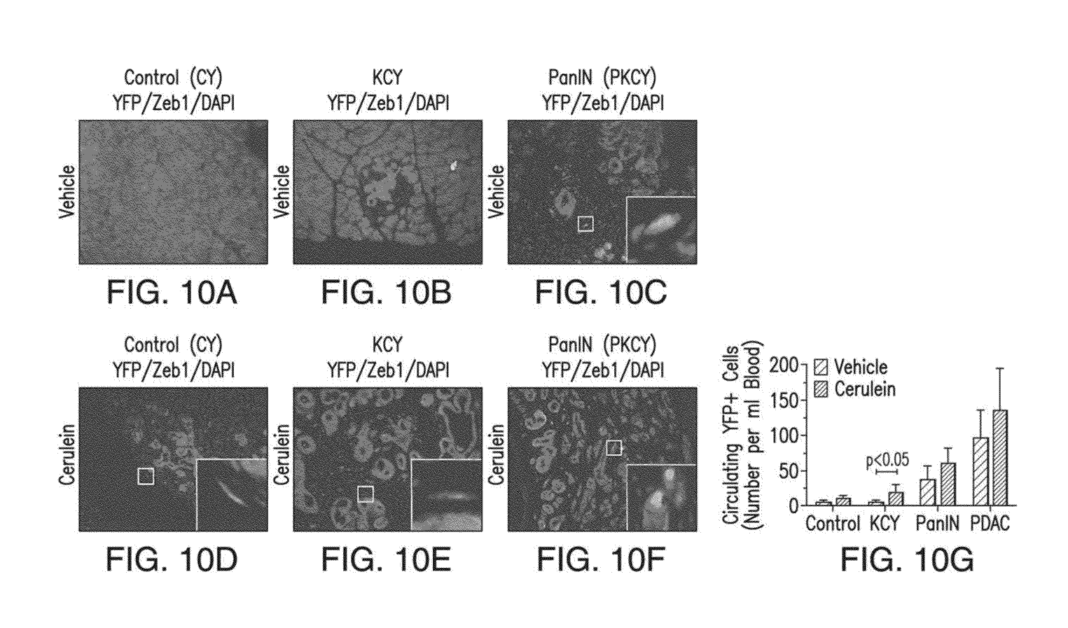

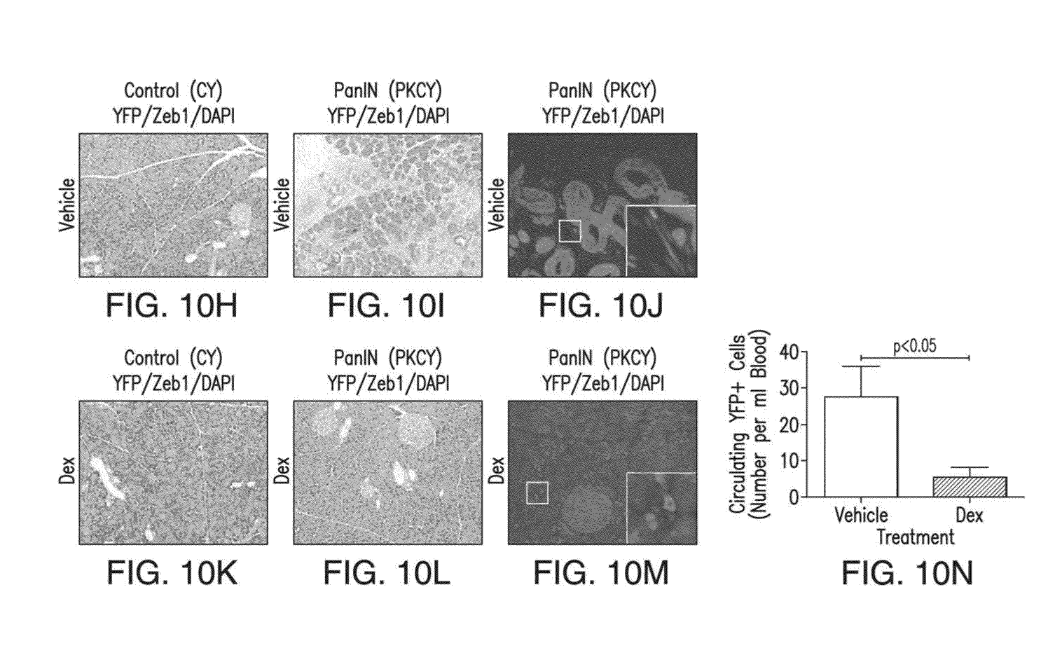

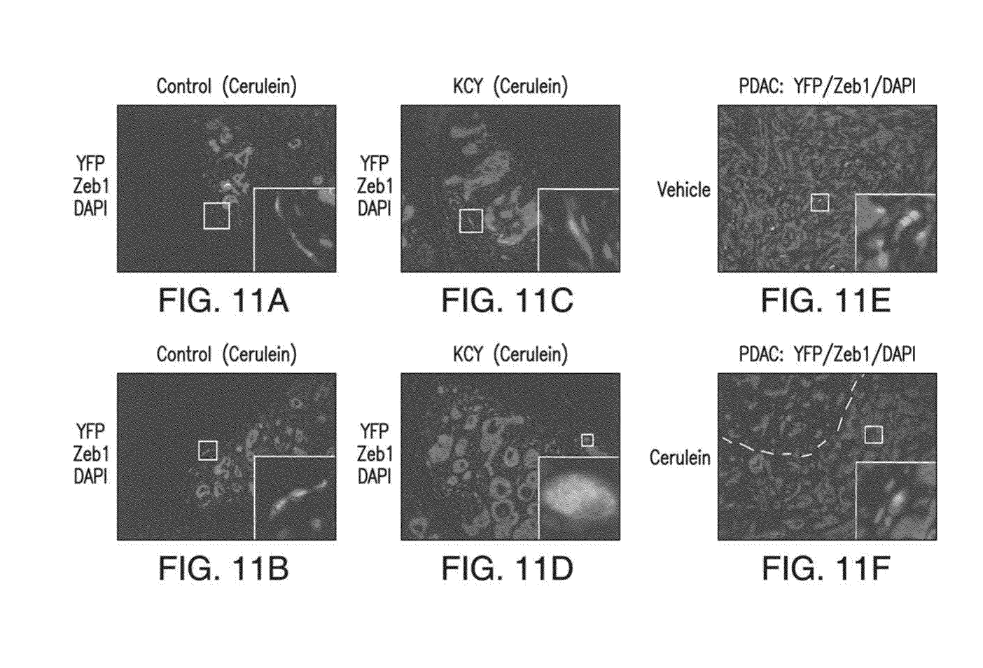

To address this possibility, a state of acute pancreatitis was induced with cerulein, a cholecystokinin analog that induces acinar cell death (Siveke et al., 2008). Initially, KCY mice were analyzed to determine whether expression of oncogenic Kras alone facilitated EMT and dissemination in response to acute pancreatitis. At 2 months of age, KCY mice had few PanIN lesions or inflammation at baseline; maximum PanIN grade found at this stage was PanIN 1 (FIG. 10B). As expected, cerulein treatment resulted in the formation of ADMIs and accelerated PanIN formation (compare FIGS. 10B and 10E; FIGS. 11A-11D). Surprisingly, cerulein treatment also resulted in a marked elevation in circulating cells, such that CPCs in 8-week-old cerulein-treated KCY mice were almost as abundant as CPCs in vehicle-treated PKCY mice of the same age (FIG. 10G). Stated otherwise, cerulein pancreatitis had nearly the same effect on CPC number as the addition of a single foxed p53 allele. Similar changes were observed in PKCY PanIN and PDAC mice treated with cerulein (FIGS. 10C, 10F, 11E, and 11F), as well as control CY animals treated with cerulein (FIGS. 10A and 10D), resulting in a significant increase in CPC number across all groups following cerulein treatment (p=0.014 by two-way ANOVA; FIG. 10G). CPCs from cerulein-treated KCY mice exhibited a nearly 100-fold enrichment of CD24⁺CD44⁺ cells compared to the source pancreas, as had been observed for PanIN CPCs, although this did not result in an increase in clonogenic growth for KCY-derived CPCs in the pancreatosphere assay (FIGS. 8B and 8C).

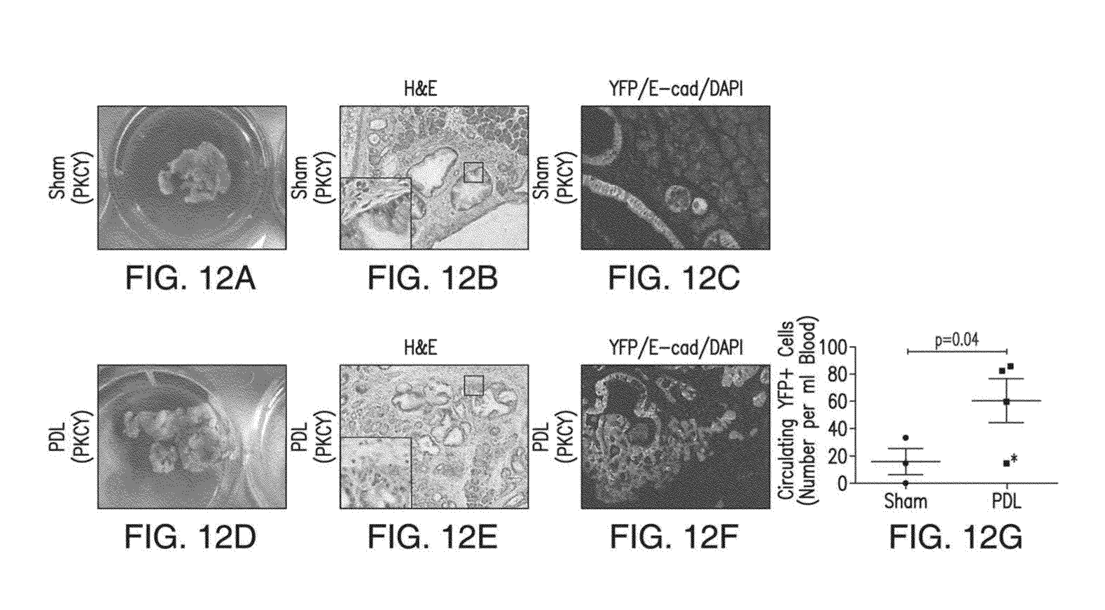

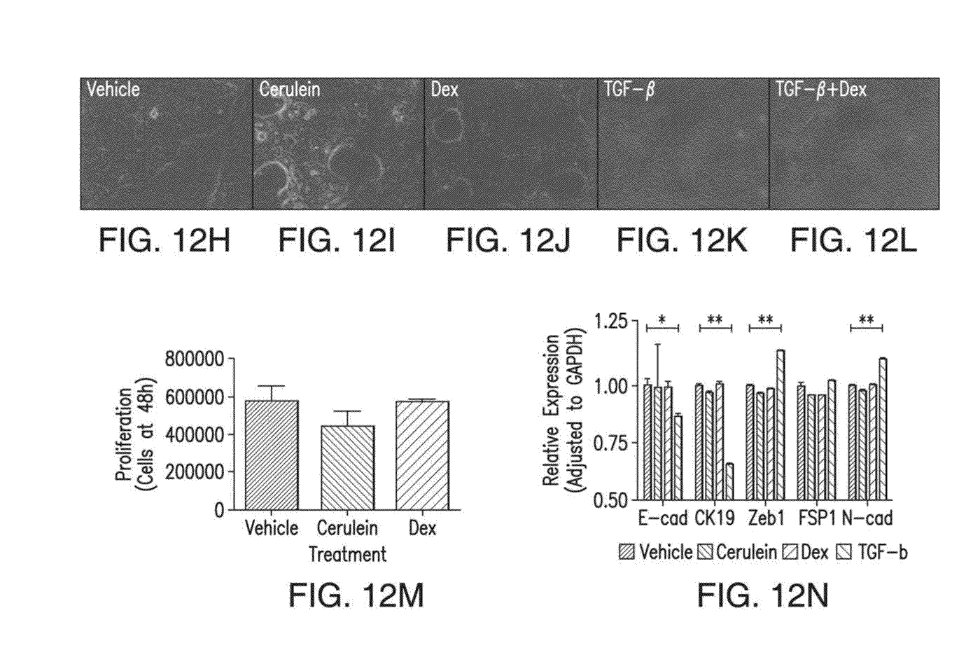

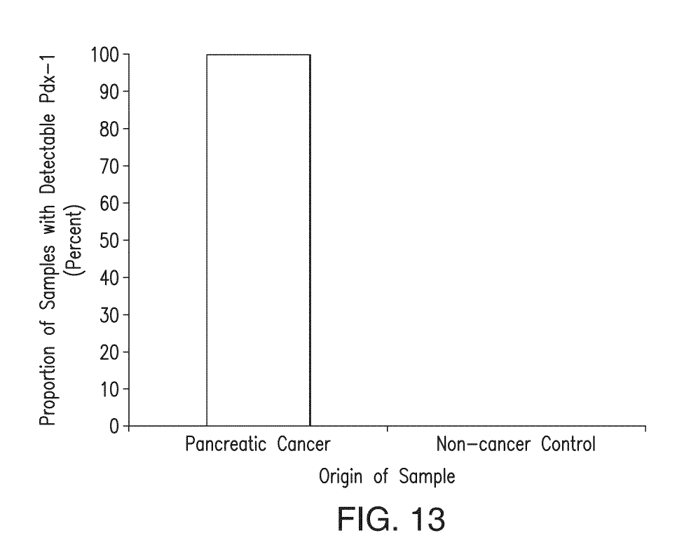

To confirm that inflammation promotes EMT and bloodstream entry, a second paradigm of pancreatitis was employed and pancreatic duct ligation (PDL) was performed on 8- to 10-week-old PKCY PanIN mice. One week after PDL, the portion of the pancreas distal to the ligation was enlarged and nodular compared to the proximal portion from the same mouse or sham-treated PanIN mice (FIGS. 12A and 12D). This resulted in inflammation, more advanced PanINs, and a loss of epithelial markers (FIGS. 12B, 12C, 12E, and 12F), as well as an increase in CPCs (FIG. 12G; p=0.042, n=7). Thus, both chemical and surgical methods for inducing pancreatitis result in an increase in EMT and CPC number.

Finally, it was determined whether inflammation is necessary for EMT and bloodstream entry. 10-week-old PKCY mice were treated with dexamethasone (Dex), a potent anti-inflammatory drug that has no effect on pancreatic histology in control animals (FIGS. 10H and 10K). As expected, vehicle treated mice at this age had PanIN lesions with an inflammatory stroma and evidence of EMT but no tumors (FIGS. 10I and 10J). Daily treatment with Dex (10 mg/kg for 7 days) resulted in a marked reduction in inflammation (FIGS. 10L and 10M; n=6 each group). Remarkably, PanINs and ADMIs were almost undetectable in these pancreata (FIGS. 10K-10M), and this change in histology was associated with a significant drop in CPC number compared to vehicle-treated controls (FIG. 10N). Importantly, neither cerulein nor Dex treatment of PanIN-derived epithelial cells in vitro had any effect on morphology, proliferation, or expression of epithelial or mesenchymal markers (FIGS. 12H-12N).

### Discussion

### Invasive Behavior Precedes Frank Tumorigenesis

Using in vivo lineage tracing, it was found that EMT, migration of epithelially derived cells into the stroma, bloodstream entry, and seeding of the liver occur at a stage of pancreatic adenocarcinoma progression previously thought to be preinvasive based on standard histological examination. The relevance of these findings to patients is supported by the detection of delaminated Pdx1⁺ cells adjacent to PanIN lesions in sections of human pancreata. Thus, this data supports a model for pancreatic cancer progression in which the seeding of distant organs occurs before, and in parallel to, tumor formation at the primary site.

Such an interpretation is especially applicable to PDAC, as the vast majority of patients with pancreatic cancer have metastatic disease at the time of diagnosis. More than 75% of patients who undergo surgical resection of small pancreatic tumors with clear surgical margins and no evidence of metastasis die from metastatic disease within 5 years (Neoptolemos et al., 2004), a finding that is consistent with early spread. Moreover, metastatic PDAC has been documented in a cohort of patients who underwent pancreatectomy for chronic pancreatitis and in whom histologic analysis of the resected pancreas revealed only PanIN lesions (Sakorafas and Sarr, 2003).

Recent genetic studies examining low-passage cell lines or microdissected primary tumors and matched metastases have concluded that metastasis is a late event in human PDAC (Campbell et al., 2010; Yachida et al., 2010). In these studies, a large proportion of mutations were shared among primary and metastatic lesions, leading to the conclusion that metastasis constituted a terminal event in the disease process. However, mathematical modeling of such phylogenetic relationships relies on assumptions about proliferation and mutation rates at stages of metastatic progression (e.g., micrometastasis) that are not measured easily. Indeed, the notion that colonization occurs early in PDAC is supported by the observation that proliferation is significantly lower in metastatic lesions (Okimura et al., 2009) compared to the primary tumor (Yachida et al., 2010), and yet their sizes at the time of diagnosis are similar.

Several lines of evidence indicate that at least some CPCs from PanIN mice (and the corresponding liver-seeding cells) are capable of giving rise to metastases. First, CPCs found in the blood of PanIN-bearing mice exhibit increased survival and self-renewal properties in vitro, suggesting that they can be able to persist for long periods of time in a foreign environment such as the liver.

Second, most PanIN-derived CPCs exhibit a YFP⁺E-cad⁻ cell surface phenotype, which our transplantation experiments showed was associated with enhanced tumor-initiating capacity. Finally, a wealth of clinical and experimental data from other systems is consistent with early spread (Weinberg, 2008). Additional experiments will be performed to confirm that cells that enter the circulation prior to the development of frank malignancy have metastatic potential. Nevertheless, the finding by routine histology that delaminating cells are indistinguishable from surrounding stromal cells indicates that current histological criteria to diagnose invasive PDAC is inadequate. These data also indicate that the location of sampling within the peripheral blood can influence the detection rate of circulating cells.

### EMT, MET, and the Acquisition of Stem Cell Characteristics

Lineage tracing enabled the distinguishing of pancreatic cells that had acquired mesenchymal characteristics from those that retained an epithelial phenotype. The majority of labeled cells that delaminated from the epithelium (i.e., locally invasive cells) expressed Zeb1, indicating that they had undergone EMT. This strong correlation between invasive behavior and the acquisition of mesenchymal characteristics in vivo suggests that EMT is not merely an epiphenomenon but rather represents a critical hurdle that cells must clear to escape from their epithelial neighbors. At present, the signals that initiate EMT in advanced PanINs and ADMIs in vivo remain to be determined.

When PDAC cells were separated according to EMT status (i.e., YFP⁺E-cad⁺ or YFP⁺E-cad⁻) and transplanted orthotopically into the pancreas, the resultant tumors were similar with respect to their epithelial and mesenchymal composition. This result demonstrates that an epithelial or mesenchymal phenotype is not a stable property of a malignant cell and provides direct evidence for a mesenchymal-to-epithelial transition (MET) in vivo. It was also noted that orthotopically transplanted YFP⁺E-cad⁻ cells from 8- to 10-week-old PKCY. PanIN mice gave rise to tumors with a much shorter latency than the same number of YFP⁺E-cad⁺ cells. One interpretation of this result is that cells that have undergone EMT at the PanIN stage are more tumorigenic; alternatively, the YFP⁺E-cad⁻ population can be enriched for a subset of cells with greater or more rapid tumor-initiating properties. In either case, our findings highlight the striking degree of plasticity that exists between epithelial and mesenchymal states during tumor progression in vivo.

In the PKCY model, cells with a CD24⁺CD44⁺ phenotype—a population possessing tumor-initiating properties by xenograft assay (Li et al., 2007)—were significantly enriched in the circulation compared to the pancreas. Accordingly, circulating cells exhibited increased survival and self-renewal in low-attachment conditions. Thus, our findings provide in vivo support for the notion that EMT is associated with the initiation of a stem cell program (Mani et al., 2008) and indicate that acquisition of a CD24⁺CD44⁺ phenotype facilitates entry into the circulation and/or survival within the bloodstream.

The development of technologies to identify circulating tumor cells (CTCs) from patients represents an enormous advance in metastasis research (Pantel et al., 2008). CTC number correlates with clinical outcome and response to chemotherapeutics in many cancers (Cristofanilli et al., 2004), and isolated disseminated cells can be interrogated for molecular changes that are associated with an increased risk of death (Stoecklein et al., 2008). CTC biology thus has both clinical utility and the potential to advance our understanding of the metastatic cascade.

However, standard techniques for isolating CTCs rely heavily upon the use of epithelial markers, particularly EpCAM, for detection (Pantel et al., 2008). The presence of EpCAM-negative YFP+ CPCs in our studies raises several possibilities: (1) standard methods that employ epithelial epitopes may not capture all CTCs; (2) cells may enter the circulation after undergoing an “incomplete” EMT, in which residual expression of EpCAM is maintained but not detected by flow cytometry; or (3) CTCs may enter the bloodstream with a mesenchymal phenotype and subsequently “revert” to an epithelial phenotype through a process of MET.

### Inflammation Promotes EMT and Dissemination

Inflammation has a well-established role in promoting tumor progression (Grivennikov et al., 2010). Using two models of pancreatitis, it has been demonstrated herein that inflammation induces EMT in CY and KCY animals and results in an increase in CPC number across all genotypes tested. Although mice bearing a single mutant Kras allele seemed to have the greatest increase in CPC number following cerulein treatment, even nonmutant pancreatic cells underwent EMT and entered the circulation in the setting of inflammation. This latter observation is quite surprising, as hematogenous spread has not been thought to occur in adult animals in the absence of a tumor. Phenotypically normal cells injected into the bloodstream can seed distant organs and persist for long periods of time until stimulated to grow (Podsypanina et al., 2008), and in both mice and humans, chronic pancreatic inflammation is strongly associated with pancreatic cancer (Grover and Syngal, 2010; Guerra et al., 2007). This study suggests that inflammation can promote cancer progression through two independent mechanisms: by facilitating changes in the microenvironment at the primary site of neoplasia and by facilitating invasion and dissemination by increasing cellular access to the circulation.

There is likely to be heterogeneity among pancreatic epithelial cells at the PanIN stage, and it is possible that bloodstream entry prior to the development of a recognizable tumor is facilitated by loss of the second allele of p53 in PKCY mice. However, the increase of CPCs in KCY and CY mice (which bear no cancer promoting mutations) following experimental pancreatitis suggests that loss of this tumor suppressor gene is not required for bloodstream entry. Interestingly, although pancreatitis augmented CPC number in KCY animals, these cells did not have the same clonal growth properties exhibited by PKCY CPCs (compare FIG. 8C with FIG. 7F), supporting the notion that p53 loss enhances the survival and/or self-renewal of circulating cells.

Finally, it has been demonstrated that the inflammatory stroma is necessary for EMT and dissemination. Treatment of 10-week old PKCY mice with dexamethasone for 1 week resulted in an almost complete elimination of PanIN lesions in the pancreas and loss of YFP+ cells from the blood, underscoring a critical requirement for the inflammatory stroma in the maintenance of premalignant PanIN lesions. Similar regression of murine PanIN disease was also seen after treatment with the cyclooxygenase inhibitor sulindac (Guerra et al., 2011).

These findings have implications for the management of individuals at high risk for pancreatic cancer, including patients with hereditary pancreatitis or kindreds with inherited pancreatic cancer. If dissemination and seeding of pancreatic epithelial cells precede the detection of a tumor in humans, as they do in the mouse model, a window of opportunity exists for prophylactic therapy in high-risk patients. Indeed, anti-inflammatory drugs have proven moderately successful at reducing mortality due to several cancers, including PDAC (Rothwell et al., 2011).

### Example 2

### Detection of Circulating Epithelial Cells as a Biomarker for Pre-Cancerous Pancreatic Cystic Lesions in Patients

### Experimental Procedures

### Patient Enrollment

Patient consent was acquired prior to enrollment and blood sampling. Upon enrollment, a basic demographic questionnaire was administered and medical data was collected from the patient or electronic medical record with patient permission. Cancer-free control patients were enrolled prior to performance of average-risk age-appropriate (age >50) colon cancer screening via colonoscopy. Exclusionary criteria for this cohort included: personal history of cancer, identification of large polyp or adenomatous pathology on previous or subsequent colonoscopy, history of any inflammatory diseases, such as inflammatory bowel disease and rheumatoid arthritis, history of abdominal surgery within the past four months. Patients with PDAC were enrolled either prior to palliative endoscopic stent placement or at the time of diagnostic EUS/FNA for a suspicious pancreatic mass. Exclusion criteria for these patients included abdominal surgery in the past four months, and personal history of other cancer or inflammatory disease. Patients with cystic lesions were enrolled prior to EUS evaluation in the pre-procedure area. Lesions that were identified by cytology of FNA as diagnostic for cancer were classified as PDAC. Patients were enrolled and analyzed if cystic lesions did not qualify for surgical resection based on Sendai criteria from CT or MR or subsequent EUS; Sendai criteria include cyst size >3 cm, main pancreatic duct involvement, septations present, cytology suspicious for dysplasia, or cysts with solid component. Further, patients were excluded from this cohort if any evidence of metastatic disease was present or if radiology interpretation prior to EUS stated that the cystic lesions was “suspicious of carcinoma” or represented “possible carcinoma”. Peripheral blood specimens were collected into Cell Save™ tubes (Johnson and Johnson, Raritan, N.J.) at the time of intravenous line placement in the pre-procedure area by nursing staff. The tube was labeled with an anonymous study identification number and overnight shipped to the Kirby laboratory at Cornell University, Ithaca, for CEC analysis within 72 h using the GEDI device.

TABLE 3
Patient Characteristics.

| | | | | | | | | | | | Size | |
|---|---|---|---|---|---|---|---|---|---|---|---|---|
| | Age | Race | Sex | FHx | BMI | Smoking | EtOH | CA19-9 | CEA | CECs | of Cyst/Tumor (mm) | Cyst type/Cancer Stage |
| Cancer- | 53 | Cauc | M | | 35.7 | Never | 0 | | | 0 | | |
| free | 40 | Cauc | M | | 26.3 | Never | 1 | | | 0 | | |
| controls | 64 | Cauc | F | | 28.3 | Never | 0 | | | 0 | | |
| n = 11 | 61 | Cauc | M | | 31.0 | Never | 1 | | | 0 | | |
| | 48 | Cauc | F | | 21.0 | Never | 0 | | | 0 | | |
| | 62 | AAM | F | | 35.4 | Never | 0 | | | 0 | | |
| | 74 | Cauc | F | | 19.8 | Never | 0 | | | 0 | | |
| | 56 | AAM | F | | 24.7 | Never | 0.5 | | | 0 | | |
| | 70 | Cauc | M | | 26.4 | Previous | 0 | | | 0 | | |
| | 51 | AAM | F | | 56.6 | Never | 0 | | | 0 | | |
| | 60 | Cauc | M | | 29.5 | Never | 0 | | | 0 | | |
| MEAN | 58.1 | | | | 30.4 | | | | | 0 | | |
| Cystic | 67 | Cauc | F | | 31.6 | Never | 0 | | | 0 | 9 | Side-branch IPMN, multiple |
| Lesion | 62 | Cauc | F | Y | 20.2 | Never | 3 | | 0 | 0 | 8 | Side-branch IPMN |
| n = 10 | 64 | Cauc | M | | 34.9 | Never | 5 | | 86.7 | 0 | 16 | MCN |
| | 75 | Cauc | M | | 24.3 | Never | 0 | | | 0 | 15 | Side-branch IPMN |
| | 65 | Cauc | F | Y | 22.4 | Never | 0 | | | 6 | 9.5 | Side-branch IPMN |
| | 60 | Cauc | M | | 28.3 | Never | 0 | 1.9 | 22 | 16 | 14 | Side-branch IPMN |
| | 72 | Cauc | M | | 21.3 | Current | 3 | | <1 | 22 | 10 | MCN |
| | 81 | Cauc | M | | 27.7 | Never | 6 | | | 0 | 15 | Side-branch IPMN |
| | 58 | Cauc | M | | 20.7 | Current | 20 | | | 0 | 5 | Side-branch IPMN |
| | 64 | Cauc | F | Y | 18.0 | Never | 5 | 46 | 3.8 | 0 | 20 | Side-branch IPMN |
| MEAN | 66.0 | | | | 25.4 | | | | | 4.4 ± 8.0 | | |
| PDAC | 92 | Cauc | M | | 27.0 | Never | 0 | 805 | 8.2 | 0 | 52 | Stage I |
| n = 9 | 65 | Cauc | M | | 33.5 | Never | 0 | | | 12 | 61 | Stage IV |
| | 76 | Cauc | M | | 29.2 | Current | 3 | 764 | 46 | 59 | 91 | Stage II |
| | 69 | Cauc | M | | 26.1 | Current | 1 | 127 | 39 | 33 | 33 | Stage IV |
| | 65 | Cauc | M | | 37.3 | Previous | 0 | 1256 | | 3 | 23 | Stage IIB |
| | 59 | AAM | M | | 24.8 | Previous | 0 | 912 | | 6 | 15 | Stage III |
| | 70 | Cauc | F | | 19.9 | Previous | 0 | 857 | | 9 | 17 | Stage IV |
| | 73 | AAM | M | | 22.0 | Never | 0 | 410 | | 0 | 40 | Stage IV |
| | 62 | Cauc | M | | 29.4 | Current | 1 | 862 | | 24 | 16 | Stage IV |
| MEAN | 69.2 | | | | 26.6 | | | | | 16.2 ± 19.5 | | |

Abbreviations:
FHx, family history;
CEA, carcinoembryonic antigen;
Cauc, Caucasian;
AAM, African-American;
IPMN, intraductal papillary mucinous neoplasm;
MCN, mucinous cyst neoplasm

### GEDI Chip Fabrication and Functionalization

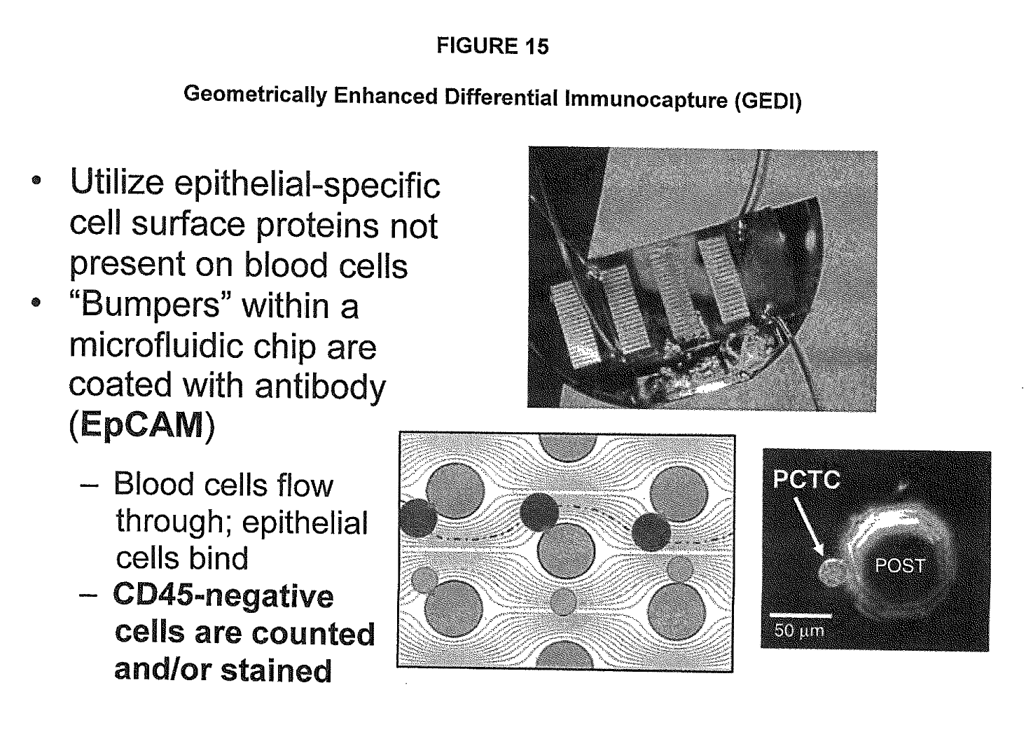

GEDI device and optimization has been described previously (Gleghorn J P, et al. (2010). Lab Chip, 10: 27-9; Kirby B J, et al. (2012) PLoS One, 7:e35976; see also FIG. 15). For this Example, the GEDI devices were obtained from AM Fitzgerald and Associates (Burlingame, Calif.) and were functionalized with biotinylated monoclonal antibodies specific to the epithelial adhesion protein, EpCAM (Santa Cruz Biotechnology, Santa Cruz) using a protocol described elsewhere (Gleghorn J P, et al. (2010). Lab Chip, 10: 27-9; Kirby B J, et al. (2012) PLoS One, 7:e35976).

GEDI Capture of CECs from Patient Blood Samples

All samples were captured and stained within 72 hours of being drawn. After mounting the GEDI devices with polymethylsiloxane (PDMS) covers, the devices were washed with 0.25 ml Phosphate-buffered saline (PBS) at a flow rate of 1 ml/hour. Cell capture was performed by flowing 1 ml patient blood through the GEDI device at 1 ml/hour. The devices were washed with 0.5 ml PBS at 1 ml/hour, until the device outlet was free from residual sample.

### Staining of Captured Cells

Cells that remained bound to the GEDI devices were fixed and stained on-chip by immersing the devices in fixative solution and incubating under gentle rocking. The samples were fixed using PHEMO fixative (3.7% formaldehyde, 0.05% glutaraldehyde, 0.5% Triton X-100 in 0.068M PIPES, 0.025M HEPES, 0.015M EGTA disodium salt, 0.003M MgCl₂-6H₂O, 10% v/v DMSO, NaOH titrated to pH 6.8) and blocked with normal goat serum. The cells were stained for CD45 using a primary (BD Biosciences, Franklin Lakes, N.J.) and a Alexa Fluoro® 488 conjugated secondary antibody (Life Technologies, Grand Island, N.Y.), cytokeratin-8 using a primary (Santa Cruz Biotechnology, Santa Cruz, Calif.) and a Alexa Fluoro® 680 conjugated secondary antibody (Life Technologies), Pdx-1 using a PE conjugated primary antibody (R&D Systems, Minneapolis, Minn.), and for nuclei using DAPI (Life Technologies). The cells were then visualized using a fluorescence microscope and image captured.

### Circulating Epithelial Cell Enumeration

CPC enumeration was performed by blinded manual inspection. A cell-like morphology with intact cytosol in reflected light, an intact, non-apoptotic DAPI+ nucleus, and CD45− surface were used as necessary identifying criteria. CK8 and Pdx-1 levels were also recorded but were not part of the enumeration protocol.

### Assessment of Pdx-1 Expression

Cell lines used for the analysis of Pdx-1 specificity included: 1) a primary murine cell line derived from sorted YFP+ cells from a lineage labeled mouse with PanIN disease and no tumor or cancer on histology (PI34; Rhim A D, et al. (2012) Cell, 148: 349-61; Agarwal B, et al. (2008) Pancreas, 36: e15-20); 2) primary human prostate cancer cell lines (LNCaP, CWR22Rv1) and 3) a primary human breast cancer cell line (MCF-7). Cell lines were stained using Alexa Fluor 647 conjugated polyclonal antibody to Pdx-1 (eBioscience, Inc., San Diego, Calif.). Briefly, cells were grown to 60-70% confluence then fixed using 4% paraformaldehyde (in PBS). After incubation with staining buffer (2% rabbit serum in PBS with 0.5% Triton X-100), cells were incubated in Pdx-1 antibody (1:200) with DAPI at room temperature, followed by washing. The stainings were visualized using a fluorescence microscope and image captured.

### Statistical Analysis

Statistical analysis was performed using Graph Pad Prism 5 and Stata 12.0 for Windows. Chi-squared tests were performed to compare the demographics of the different patient groups. For comparisons of circulating epithelial cell values across patient groups, the Mann-Whitney U-test for non-parametric data was performed. For comparison of circulating epithelial cell values among all patient groups, a one-way ANOVA analysis was performed with the cancer-free control group designated as the negative control group.

### Results

### Geometrically Enhanced Differential Immunocapture (GEDI) of Circulating Epithelial Cells.

A blinded prospective pilot study of three patient groups was performed: 1) patients with no history of cancer presenting for average-risk, age-appropriate colonoscopy screening and no adenomas detected; 2) patients with precancerous cystic lesions (intraductal papillary mucinous neoplasm (IPMN) or mucinous cystic neoplasms (MCN)) of the pancreas with no evidence of tumor or metastasis on CT or MRI, who did not qualify for surgery under Sendai criteria (Tanaka M, et al. (2006) Pancreatology, 6: 17-32; including no evidence of dysplasia or cancer on FNA, if done); and 3) patients with cytology-confirmed PDAC. Peripheral blood was obtained from consented patients prior to procedure.

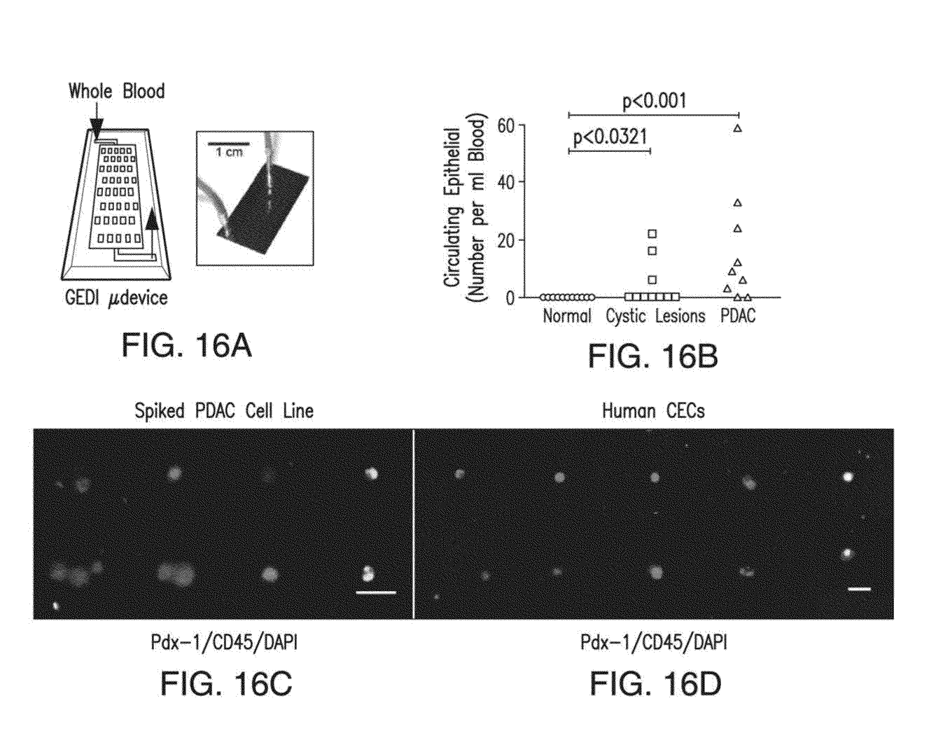

Patient blood samples were analyzed using geometrically enhanced differential immunocapture (GEDI), a microfluidic platform that has been shown to detect circulating tumor cells from patients with prostate cancers with high sensitivity (Gleghorn J P, et al. (2010). Lab Chip, 10: 27-9; Kirby B J, et al. (2012) PLoS One, 7:e35976; see also FIG. 15). In this Example, the GEDI chip was functionalized using antibodies to epithelial cell adhesion molecule, EpCAM, to capture circulating epithelial cells (CECs) (FIG. 16A). Captured cells were then stained with fluorescently conjugated antibodies to CD45, a universal marker of leukocytes, and pancreas and duodenal homeobox protein-1 (Pdx-1), a pancreas-specific transcription factor, and imaged using a fluorescence microscope. Captured cells were considered circulating epithelial cells if they were CD45− and DAPI+.

In this blind study, 11 control patients, 10 patients with cystic lesions, and 9 patients with pancreatic duct adenocarcinoma (PDAC) were enrolled (Table 3). Cyst- and cancer-free patients tended to be younger compared with cystic lesion and PDAC patients (p=0.016 by Chi-squared test); however, there were no differences in other demographics. Most (80%) cystic lesions were classified as side-branch IPMNs and ranged in size from 5 to 20 mm. Patients with PDAC had a wide range in primary tumor diameter (15-91 mm) and tumor stage (I to IV).

In the normal control patients, no CECs were detected, whereas 7 of the 9 (78%) PDAC patients had detectable CECs, with an average of 16.2±19.5 CEC/ml blood (p<0.001 compared to the control group by Mann-Whitney Test) (FIG. 16B). 3 of the 10 (30%) patients with cystic lesions had detectable CECs, averaging 4.4±8.0 CECs/ml blood (p=0.0321 compared to the control group by Mann-Whitney Test), and there was a significant difference in CECs detected across the three groups by one-way ANOVA analysis (p=0.018) (FIG. 16B). No correlation between CEC count and tumor size once a tumor has been detected, cancer stage or serum CA19-9 and CEA was found; however, the data indicates that the CEC count correlates with cancer progression with respect to the presence of an early cancer focus that cannot be detected versus a large tumor that is associated with late stage cancer.

Additional analyses were performed on captured CECs. To confirm the pancreas origin of CECs, captured cells were stained for Pdx-1, a pancreas-specific transcription factor, expressed in up to 60% of all CECs in mouse models of PDAC (Rhim A D, et al. (2012) Cell, 148: 349-61). Adherent and GEDI-captured primary PDAC cells also expressed nuclear Pdx-1 (21% of PI34 and 10.7% of Panc-01; FIG. 16C). However, no nuclear Pdx-1 was detected within human breast (MCF-7) or prostate (LNCaP, CWR22Rv1) cancer cells or CD45+ leukocytes. These data suggest that Pdx-1 is a specific marker of pancreas-derived cells. In the current study, 29% of all CECs exhibited nuclear Pdx-1 staining (FIG. 16D). These data confirm that at least a portion of all GEDI-captured epithelial cells derive from the pancreas.

### Discussion

A widely-accepted paradigm in cancer biology is that epithelial cancers progress in a linear manner, whereby cancer-defining properties are acquired sequentially (Weinberg R A. (2008) Carcinogenesis, 29:1092-5). In this model, cancer cells acquire metastatic potential after large primary tumors are established. However, in pancreatic ductal adenocarcinoma (PDAC), the linear progression model cannot be reconciled with clinical observations. A number of patients undergoing pancreatectomy for chronic pancreatitis will develop disseminated PDAC although no tumors and only precancerous pancreatic intraepithelial neoplasias (PanINs) are found on histologic analysis (Sakorafas G H, et al. (2003) Dig. Liver Dis., 35: 482-5). Additionally, in patients with small primary tumors (<2 cm), who have no clinical evidence of metastatic disease, five year survival after pancreatectomy is <18% due to recurrent metastatic disease (Agarwal B, et al. (2008) Pancreas, 36: e15-20). These data indicate that metastatic seeding occurs before the formation of large primary tumors. Moreover, a recent study showed that hematogenous dissemination occurs prior to tumor formation in a lineage-labeled genetic model of PDAC, at which time the pancreas contained only PanIN (Rhim A D, et al. (2012) Cell, 148: 349-61). Based on the clinical characteristics of PDAC and the findings within a recapitulative mouse model as described in Example 1, blood-borne dissemination can occur in patients with pre-cancerous lesions of the pancreas in the absence of clinically detectable tumors.

Using state-of-the-art microfluidic technology (Kirby B J, et al. (2012) PLoS One, 7:e35976) and immunofluorescence staining, it was determined that pancreas epithelial cells can enter the bloodstream in patients with cystic lesions of the pancreas prior to the clinical diagnosis of cancer. Specifically, CECs were captured in 30% of patients with precancerous cystic lesions (Sendai criteria negative), 78% with PDAC and 0% of controls. These findings indicate that the ability to seed the bloodstream can precede the formation of detectable tumors, supporting the findings observed in genetic mouse models of PDAC (Rhim A D, et al. (2012) Cell, 148: 349-61 and Example 1). These data are further supported by the recent finding that 24.6% of resected side-branch IPMNs that do not satisfy Sendai criteria, contain regions of high-grade dysplasia or invasive carcinoma (Fritz S, et al. (2012) Ann. Surg., 256: 313-20). Data from the PDAC mouse model predict that these cells represent early, occult cancer cells (Rhim A D, et al. (2012) Cell, 148: 349-61).

These findings have significant implications for the use of circulating epithelial cell detection as a biomarker for cancer risk assessment in patients at risk for PDAC.

### Example 3

### Detection of Circulating Epithelial Cells as a Biomarker for Gastrointestinal Inflammation in Patients

A clinical study of circulating epithelial cells (CECs) in patients with inflammatory bowel diseases (IBD) is conducted to examine the utility of CECs as biomarkers for inflammation and their use in assessing the efficacy of therapy in human patients. The patient population to be enrolled are those with an IBD who are receiving ongoing treatment. Patients have their blood drawn at the beginning of the study, and periodically during treatment. Peripheral venous blood specimens are drawn from patients into CellSave tubes and analyzed by the method described in Example 2 within 72 hours of collection.

A custom, optimized version of the GEDI microfluidic platform is used to detect circulating epithelial cells from the patient specimens. The details of the GEDI platform is published elsewhere (Gleghorn et al., 2009), and is summarized in Example 2. In these experiments, the device is customized with antibodies specific for epithelial cell adhesion molecule (EpCAM). After functionalization of the GEDI chip, 1 ml of patient blood is circulated through the chip at a rate of 1 mL/hour, with the residual blood being recirculated in multiple washes. Cells that are bound to the chip are then fixed with formaldehyde. GEDI-captured cells are then stained with antibodies to CD45 (leukocyte marker), cytokeratin 8 (another epithelial-specific marker), a marker specific to intestine or colon epithelial cells, as well as DAPI, a marker for nucleated cells. The cells are then quantified and photographed using an upright multicolor immunofluorescence microscope. Cells are considered circulated intestinal or colonic epithelial cell if they are DAPI+, CD45−, cytokeratin 8+, and if they are positive for the intestinal or colonic marker. As the patient receives treatment, additional blood samples are drawn from the patient and the method is repeated to obtain additional CEC counts for the patient.

A decrease in CECs from the colon indicates a decrease in inflammation in the colon, and an increase in CECs from the colon is indicative of increased inflammation in the colon.

### REFERENCES

Aguirre, A. J., Bardeesy, N., Sinha, M., Lopez, L., Tuveson, D. A., Homer, J., Redston, M. S., and DePinho, R. A. (2003). Activated Kras and Ink4a/Arf deficiency cooperate to produce metastatic pancreatic ductal adenocarcinoma. Genes Dev 17, 3112-3126.
Bardeesy, N., Aguirre, A. J., Chu, G. C., Cheng, K. H., Lopez, L. V., Hezel, A. F., Feng, B., Brennan, C., Weissleder, R., Mahmood, U., et al. (2006). Both p16(Ink4a) and the p19(Arf)-p53 pathway constrain progression of pancreatic adenocarcinoma in the mouse. Proc Nati Acad Sci USA 103, 5947-5952.
Brabletz, S., Bajdak, K., Meidhof, S., Burk, U., Niedermann, G., Firat, E., Wellner, U., Dimmler, A., Faller, G., Schubert, J., et al. (2011). The ZEB1/miR-200 feedback loop controls Notch signalling in cancer cells. EMBO J 30, 770-782.
Cairns, J. (1975). Mutation selection and the natural history of cancer. Nature 255, 197-200.
Campbell, P. J., Yachida, S., Mudie, L. J., Stephens, P. J., Pleasance, E. D., Stebbings, L. A., Morsberger, L. A., Latimer, C., McLaren, S., Lin, M L, et al. (2010). The patterns and dynamics of genomic instability in metastatic pancreatic cancer. Nature 467, 1109-1113.
Clark, C. E., Hingorani, S. R., Mick, R., Combs, C., Tuveson, D. A., and Vonderheide, R. H. (2007). Dynamics of the immune reaction to pancreatic cancer from inception to invasion. Cancer Res 67, 9518-9527.
Coussens, L. M., and Werb, Z. (2002). Inflammation and cancer. Nature 420, 860-867.
Cristofanilli, M., Budd, G. T., Ellis, M. J., Stopeck, A., Matera, J., Miller, M. C., Reuben, J. M., Doyle, G. V., Allard, W. J., Terstappen, L. W., et al. (2004). Circulating tumor cells, disease progression, and survival in metastatic breast cancer. N Engl J Med 351, 781-791.
Gleghorn et al, (2009). Capture of circulating tumor cells from whole blood of prostate cancer patients using geometrically enhanced differential immunocapture (GEDI) and a prostate-specific antibody. Lab Chip 10:27-29.
Greco, F. A., and Hainsworth, J. D. (2009). Introduction: unknown primary cancer. Semin Oncol 36, 6-7.
Grivennikov, S. I., Greten, F. R., and Karin, M. (2010). Immunity, inflammation, and cancer. Cell 140, 883-899.
Grover, S., and Syngal, S. (2010). Hereditary pancreatic cancer. Gastroenterology 139, 1076-1080, 1080 e1071-1072.
Gu, G., Brown, J. R., and Melton, D. A. (2003). Direct lineage tracing reveals the ontogeny of pancreatic cell fates during mouse embryogenesis. Mech Dev 120, 35-43.
Guerra, C., Collado, M., Navas, C., Schuhmacher, A. J., Hernandez-Porras, I., Canamero, M., Rodriguez-Justo, M., Serrano, M., and Barbacid, M. (2011). Pancreatitis-induced inflammation contributes to pancreatic cancer by inhibiting oncogene-induced senescence. Cancer Cell 19, 728-739.
Guerra, C., Schuhmacher, A. J., Canamero, M., Grippo, P. J., Verdaguer, L., Perez-Gallego, L., Dubus, P., Sandgren, E. P., and Barbacid, M. (2007). Chronic pancreatitis is essential for induction of pancreatic ductal adenocarcinoma by K-Ras oncogenes in adult mice. Cancer Cell 11, 291-302.
Gupta, P. B., Mani, S., Yang, J., Hartwell, K., and Weinberg, R. A. (2005). The evolving portrait of cancer metastasis. Cold Spring Harb Symp Quant Biol 70, 291-297.
Habbe, N., Shi, G., Meguid, R. A., Fendrich, V., Esni, F., Chen, H., Feldmann, G., Stoffers, D. A., Konieczny, S. F., Leach, S. D., et al. (2008). Spontaneous induction of murine pancreatic intraepithelial neoplasia (mPanIN) by acinar cell targeting of oncogenic Kras in adult mice. Proc Nati Acad Sci USA 105, 18913-18918.
Hanahan, D., and Weinberg, R. A. (2011). Hallmarks of cancer: the next generation. Cell 144, 646-674.
Hellman, S. (1994). Karnofsky Memorial Lecture. Natural history of small breast cancers. J Clin Oncol 12, 2229-2234.
Hermann, P. C., Huber, S. L., Herrler, T., Aicher, A., Ellwart, J. W., Guba, M., Bruns, C. J., and Heeschen, C. (2007). Distinct populations of cancer stem cells determine tumor growth and metastatic activity in human pancreatic cancer. Cell Stem Cell 1, 313-323.
Bezel, A. F., Kimmelman, A. C., Stanger, B. Z., Bardeesy, N., and Depinho, R. A. (2006). Genetics and biology of pancreatic ductal adenocarcinoma. Genes Dev 20, 1218-1249.
Hingorani, S. R., Petricoin, E. F., Maitra, A., Rajapakse, V., King, C., Jacobetz, M. A., Ross, S., Conrads, T. P., Veenstra, T. D., Hitt, B. A., et al. (2003). Preinvasive and invasive ductal pancreatic cancer and its early detection in the mouse. Cancer Cell 4, 437-450.
Husemann, Y., Geigl, J. B., Schubert, F., Musiani, P., Meyer, M., Burghart, E., Formi, G., Eils, R., Fehm, T., Riethmuller, G., et al. (2008). Systemic spread is an early step in breast cancer. Cancer Cell 13, 58-68.
Ignatiadis, M., Rothe, F., Chaboteaux, C., Durbecq, V., Rouas, G., Criscitiello, C., Metallo, J., Kheddoumi, N., Singhal, S. K., Michiels, S., et al. (2011). HER2-positive circulating tumor cells in breast cancer. PLoS One 6, e15624.
Jimeno, A., Feldmann, G., Suarez-Gauthier, A., Rasheed, Z., Solomon, A., Zou, G. M., Rubio-Viqueira, B., Garcia-Garcia, E., Lopez-Rios, F., Matsui, W., et al. (2009). A direct pancreatic cancer xenograft model as a platform for cancer stem cell therapeutic development. Mol Cancer Ther 8, 310-314.
Klein, C. A. (2009). Parallel progression of primary tumours and metastases. Nat Rev Cancer 9, 302-312.
Ledford, H. (2011). Cancer theory faces doubts. Nature 472, 273.
Li, C., Heidt, D. G., Dalerba, P., Burant, C. F., Zhang, L., Adsay, V., Wicha, M., Clarke, M. F., and Simeone, D. M. (2007). Identification of pancreatic cancer stem cells. Cancer Res 67, 1030-1037.
Mani, S. A., Guo, W., Liao, M. J., Eaton, E. N., Ayyanan, A., Zhou, A. Y., Brooks, M., Reinhard, F., Zhang, C. C., Shipitsin, M., et al. (2008). The epithelial-mesenchymal transition generates cells with properties of stem cells. Cell 133, 704-715.
Mohammad, R. M., Dugan, M. C., Mohamed, A. N., Almatchy, V. P., Flake, T. M., Dergham, S. T., Shields, A. F., Al-Katib, A. A., Vaitkevicius, V. K., and Sarkar, F. H. (1998). Establishment of a human pancreatic tumor xenograft model: potential application for preclinical evaluation of novel therapeutic agents. Pancreas 16, 19-25.
Moody, S. E., Perez, D., Pan, T. C., Sarkisian, C. J., Portocarrero, C. P., Sterner, C. J., Notorfrancesco, K. L., Cardiff, R. D., and Chodosh, L. A. (2005). The transcriptional repressor Snail promotes mammary tumor recurrence. Cancer Cell 8, 197-209.
Neoptolemos, J. P., Slacken, D. D., Friess, H., Bassi, C., Dunn, J. A., Hickey, H., Beger, H., Fernandez-Cruz, L., Dervenis, C., Lacaine, F., et al. (2004). A randomized trial of chemoradiotherapy and chemotherapy after resection of pancreatic cancer. N Engl J Med 350, 1200-1210.
Okimura, A., Hirano, H., Nishigami, T., Ucyama, S., Tachibana, S., Fukuda, Y., Yamanegi, K., Ohyama, H., Terada, N., and Nakasho, K. (2009). Immunohistochemical analyses of E-cadherin, beta-catenin, CD44s, and CD44v6 expressions, and Ki-67 labeling index in intraductal papillary mucinous neoplasms of the pancreas and associated invasive carcinomas. Med Mol Morphol 42, 222-229.
Pantel, K., Brakenhoff, R. H., and Brandt, B. (2008). Detection, clinical relevance and specific biological properties of disseminating tumour cells. Nat Rev Cancer 8, 329-340.
Park, J. Y., Hong, S. M., Klimstra, D. S., Goggins, M. G., Maitra, A., and Hruban, R. H. (2011). Pdx1 Expression in Pancreatic Precursor Lesions and Neoplasms. Appl Immunohistochem Mol Morphol.
Podsypanina, K., Du, Y. C., Jechlinger, M., Beverly, L. J., Hambardzumyan, D., and Varmus, H. (2008). Seeding and propagation of untransformed mouse mammary cells in the lung. Science 321, 1841-1844.
Polyak, K., and Weinberg, R. A. (2009). Transitions between epithelial and mesenchymal states: acquisition of malignant and stem cell traits. Nat Rev Cancer 9, 265-273.
Rothwell, P. M., Fowkes, F. G., Belch, J. F., Ogawa, H., Warlow, C. P., and Meade, T. W. (2011). Effect of daily aspirin on long-term risk of death due to cancer: analysis of individual patient data from randomised trials. Lancet 377, 31-41.
Rovira, M., Scott, S. G., Liss, A. S., Jensen, J., Thayer, S. P., and Leach, S. D. (2010). Isolation and characterization of centroacinar/terminal ductal progenitor cells in adult mouse pancreas. Proc Natl Acad Sci USA 107, 75-80.
Sakorafas, G. H., and San, M. G. (2003). Pancreatic cancer after surgery for chronic pancreatitis. Dig Liver Dis 35, 482-485.
Sanger, N., Effenberger, K. E., Riethdorf, S., van Haasteren, V., Gauwerky, J., Wiegratz, I., Strebhardt, K., Kaufmann, M., and Pantel, K. (2011). Disseminated tumor cells in the bone marrow of patients with ductal carcinoma in situ. Int J. Cancer.
Siveke, J. T., Lubeseder-Martellato, C., Lee, M., Mazur, P. K., Nakhai, H., Radtke, F., and Schmid, R. M. (2008). Notch signaling is required for exocrine regeneration after acute pancreatitis. Gastroenterology 134, 544-555.
Stairs, D. B., Bayne, L. J., Rhoades, B., Vega, M. E., Waldron, T. J., Kalabis, J., Klein-Szanto, A., Lee, J. S., Katz, J. P., Diehl, J. A., et al. (2011). Deletion of p120-Catenin Results in a Tumor Microenvironment with Inflammation and Cancer that Establishes It as a Tumor Suppressor Gene. Cancer Cell 19, 470-483.
Stoecklein, N. H., Hosch, S. B., Bezler, M., Stern, F., Hartmann, C. H., Vay, C., Siegmund, A., Scheunemann, P., Schurr, P., Knoefel, W. T., et al. (2008). Direct genetic analysis of single disseminated cancer cells for prediction of outcome and therapy selection in esophageal cancer. Cancer Cell 13, 441-453.
Trimboli, A. J., Fukino, K., de Bruin, A., Wei, G., Shen, L., Tanner, S. M., Creasap, N., Rosol, T. J., Robinson, M. L., Eng, C., et al. (2008). Direct evidence for epithelial-mesenchymal transitions in breast cancer. Cancer Res 68, 937-945.
Wang, F., Sloss, C., Zhang, X., Lee, S. W., and Cusack, J. C. (2007). Membrane-bound heparin-binding epidermal growth factor like growth factor regulates E-cadherin expression in pancreatic carcinoma cells. Cancer Res 67, 8486-8493.
Weinberg, R. A. (2008). Mechanisms of malignant progression. Carcinogenesis 29, 1092-1095.
Yachida, S., Jones, S., Bozic, I., Antal, T., Leary, R., Fu, B., Kamiyama, M., Hruban, R. H., Eshleman, J. R., Nowak, M. A., et al. (2010). Distant metastasis occurs late during the genetic evolution of pancreatic cancer. Nature 467, 1114-1117.

Various publications, GenBank Accession Nos., patents, and patent applications are cited herein, the contents of which are hereby incorporated by reference in their entireties.

1. A method of assessing whether a subject has or is at risk for developing an epithelial cancer, comprising:
(a) determining the presence of one or more circulating epithelial cells in a biological fluid sample obtained from the subject,
wherein the presence of the circulating epithelial cells in the biological fluid sample is an indication that the subject has or is at risk for developing an epithelial cancer.

2. The method of claim 1, wherein the epithelial cancer is selected from the group consisting of pancreatic cancer, kidney cancer, renal cell carcinoma, urogenital cancer, urothelial carcinoma of the urinary bladder, urothelial carcinoma of the kidney, urothelial carcinoma of the renal pelvis, urothelial carcinoma of the ureter, prostate cancer, lung carcinoma, non-small cell lung carcinoma, small cell lung carcinoma, neuroendocrine carcinoma, carcinoid tumor, breast carcinoma, ductal breast carcinoma, lobular breast carcinoma, mixed ductal and lobular breast carcinoma, thyroid carcinoma, papillary thyroid carcinoma, follicular thyroid carcinoma, medullary thyroid carcinoma, brain cancer, meningioma, astrocytoma, glioblastoma, cerebellum tumors, medulloblastoma, ependymoma, ovarian carcinoma, ovarian serous carcinoma, ovarian mucinous carcinoma, ovarian endometrioid carcinoma, cervical cancer, cervical squamous cell carcinoma, invasive squamous cell cervical carcinoma, endocervical cervical adenocarcinoma, uterine endometrial carcinoma, uterine endometrioid carcinoma, uterine serous carcinoma, uterine mucinous carcinoma, primary peritoneal carcinoma, mesothelioma, pleura mesothelioma, peritoneum mesothelioma, eye cancer, retinoblastoma, muscle cancer, sarcoma, rhapdomyosarcoma, leiomyosarcoma, lymphoma, esophageal cancer, esophageal adenocarcinoma, esophageal squamous cell carcinoma, gastric cancer, gastric adenocarcinoma, gastrointestinal stromal tumor, liver cancer, hepatocellular carcinoma, bile duct cancer, small intestinal cancer, small intestinal stromal cancer, carcinoid tumor, colon cancer, adenocarcinoma of the colon, colon high grade dysplasia, colon carcinoid tumor, testicular cancer, skin cancer, melanoma, squamous cell carcinoma and adrenal carcinoma.

3. The method of claim 1, wherein the cancer is selected from the group consisting of pancreatic cancer, breast cancer, lung cancer, liver cancer, hepatocellular carcinoma, cholangiocarcinoma, esophageal cancer, stomach cancer, colon cancer, rectum cancer, prostate cancer, uterine cancer, kidney cancer and bladder cancer.

4. The method of claim 2, further comprising identifying the type of epithelial cancer by determining the presence of a tissue-specific biomarker on the circulating epithelial cells.

5. The method of claim 4, wherein the tissue-specific biomarker is Pdx-1.

6. The method of claim 1, wherein the method is carried out prior to the identification of a primary tumor in the subject.

7. The method of claim 5, wherein the presence of Pdx-1 is detected using a reagent which specifically binds to the Pdx-1 protein.

8. The method of claim 7, wherein the reagent is selected from the group consisting of an antibody, an antibody derivative, an antigen-binding antibody fragment, a non-antibody peptide which specifically binds the Pdx-1 protein, as well as combinations thereof.

9. The method of claim 5, wherein the presence of Pdx-1 further indicates a subject has pancreatic cancer that has metastasized or is likely to metastasize.

10. A method of assessing the efficacy of a therapy for preventing or treating an epithelial cancer in a subject, the method comprising:
(a) determining the level of circulating epithelial cells in a biological fluid sample obtained from the subject, prior to therapy; and
(b) determining the level of circulating epithelial cells in a biological fluid sample obtained from the subject during the therapy,
wherein the therapy is efficacious for preventing or treating an epithelial cancer in the subject when there is a lower level of circulating epithelial cells in the sample obtained during therapy, relative to the sample obtained prior to therapy.

11. The method of claim 10, wherein the epithelial cancer is selected from the group consisting of pancreatic cancer, kidney cancer, renal cell carcinoma, urogenital cancer, urothelial carcinoma of the urinary bladder, urothelial carcinoma of the kidney, urothelial carcinoma of the renal pelvis, urothelial carcinoma of the ureter, prostate cancer, lung carcinoma, non-small cell lung carcinoma, small cell lung carcinoma, neuroendocrine carcinoma, carcinoid tumor, breast carcinoma, ductal breast carcinoma, lobular breast carcinoma, mixed ductal and lobular breast carcinoma, thyroid carcinoma, papillary thyroid carcinoma, follicular thyroid carcinoma, medullary thyroid carcinoma, brain cancer, meningioma, astrocytoma, glioblastoma, cerebellum tumors, medulloblastoma, ependymoma, ovarian carcinoma, ovarian serous carcinoma, ovarian mucinous carcinoma, ovarian endometrioid carcinoma, cervical cancer, cervical squamous cell carcinoma, invasive squamous cell cervical carcinoma, endocervical cervical adenocarcinoma, uterine endometrial carcinoma, uterine endometrioid carcinoma, uterine serous carcinoma, uterine mucinous carcinoma, primary peritoneal carcinoma, mesothelioma, pleura mesothelioma, peritoneum mesothelioma, eye cancer, retinoblastoma, muscle cancer, sarcoma, rhapdomyosarcoma, leiomyosarcoma, lymphoma, esophageal cancer, esophageal adenocarcinoma, esophageal squamous cell carcinoma, gastric cancer, gastric adenocarcinoma, gastrointestinal stromal tumor, liver cancer, hepatocellular carcinoma, bile duct cancer, small intestinal cancer, small intestinal stromal cancer, carcinoid tumor, colon cancer, adenocarcinoma of the colon, colon high grade dysplasia, colon carcinoid tumor, testicular cancer, skin cancer, melanoma, squamous cell carcinoma and adrenal carcinoma.

12. The method of claim 10, wherein the cancer is selected from the group consisting of pancreatic cancer, breast cancer, lung cancer, liver cancer, hepatocellular carcinoma, cholangiocarcinoma, esophageal cancer, stomach cancer, colon cancer, rectum cancer, prostate cancer, uterine cancer, kidney cancer and bladder cancer.

13. The method of claim 11, further comprising identifying the type of epithelial cancer by determining the presence of a tissue-specific biomarker on the circulating epithelial cells.

14. The method of claim 13, wherein the tissue-specific biomarker is Pdx-1.

15. The method of claim 14, wherein the presence of Pdx-1 is detected using a reagent which specifically binds to the Pdx-1 protein.

16. The method of claim 15, wherein the reagent is selected from the group consisting of an antibody, an antibody derivative, an antigen-binding antibody fragment, a non-antibody peptide which specifically binds the Pdx-1 protein, as well as combinations thereof.

17. A method of diagnosing an inflammatory disease in a subject, the method comprising determining the presence of one or more circulating epithelial cells in a biological fluid sample obtained from the subject, wherein the presence of the one or more circulating epithelial cells is an indication that the subject is afflicted with an inflammatory disease.

18. The method of claim 17, wherein the determination of the level of circulating epithelial cells is accomplished by detecting the expression of Pdx-1 in the cells of the biological fluid sample.

19. The method of claim 18, wherein the presence of Pdx-1 is detected using a reagent which specifically binds to the Pdx-1 protein.

20. The method of claim 19, wherein the reagent is selected from the group consisting of an antibody, an antibody derivative, an antigen-binding antibody fragment, a non-antibody peptide which specifically binds the Pdx-1 protein, as well as combinations thereof.

21. The method of claim 17, wherein the inflammatory disease is a gastrointestinal inflammatory disease.

22. A method of assessing the efficacy of a therapy for an inflammatory disease in a subject, the method comprising:
(a) determining the level of circulating epithelial cells in a biological fluid sample obtained from the subject prior to initiation of therapy for the inflammatory disease; and
(b) determining the level of circulating epithelial cells in a biological fluid sample obtained from the subject at one of more time points during therapy,
wherein the therapy is efficacious for treating or decreasing inflammation in the subject when there is a lower level of circulating epithelial cells in the sample obtained during therapy, relative to the sample obtained prior to therapy.

23. The method of claim 22, wherein the inflammatory disease is a gastrointestinal inflammatory disease.

24. The method of claim 22, wherein the determination of the level of circulating epithelial cells is accomplished by detecting the expression of Pdx-1 in the cells of the biological fluid sample.

25. The method of claim 24, wherein the presence of Pdx-1 is detected using a reagent which specifically binds to the Pdx-1 protein.

26. The method of claim 25, wherein the reagent is selected from the group consisting of an antibody, an antibody derivative, an antigen-binding antibody fragment, a non-antibody peptide which specifically binds the Pdx-1 protein, as well as combinations thereof.

27. A kit for assessing whether a subject is at risk for developing an epithelial cancer, comprising a reagent that specifically binds to an epithelial tissue-specific biomarker in circulating epithelial cells present in a biological fluid sample.
